(19) United States
(12) Patent Application Publication
(10) Pub. No.: US 2011/0198582 A1
(43) Pub. Date: Aug. 18, 2011

(54) ORGANIC LIGHT-EMITTING DEVICE

(75) Inventors: Takayuki Horiuchi, Tokyo (JP); Takeshi Sekiguchi, Tokyo (JP); Jun Kamatani, Tokyo (JP); Akihito Saitoh, Yokohama-shi (JP); Naoki Yamada, Inagi-shi (JP); Tetsuya Kosuge, Yokohama-shi (JP)

(73) Assignee: CANON KABUSHIKI KAISHA, Tokyo (JP)

(21) Appl. No.: 13/125,020

(22) PCT Filed: Oct. 19, 2009
(86) PCT No.: PCT/JP2009/068299

§ 371 Date: Apr. 19, 2011

(30) Foreign Application Priority Data
Oct. 22, 2008 (JP) 2008-272133
Sep. 11, 2009 (JP) 2009-210707

(51) Int. Cl. H01L 51/54

(52) U.S. Cl. 257/40; 257/E51.024

(57) ABSTRACT

An organic light-emitting device includes an organic compound layer containing an organic compound in which the 3-position of a benzo[k]fluoranthene ring is bonded to the 8-position of a fluoranthene ring, and an organic compound having a pyrene ring.

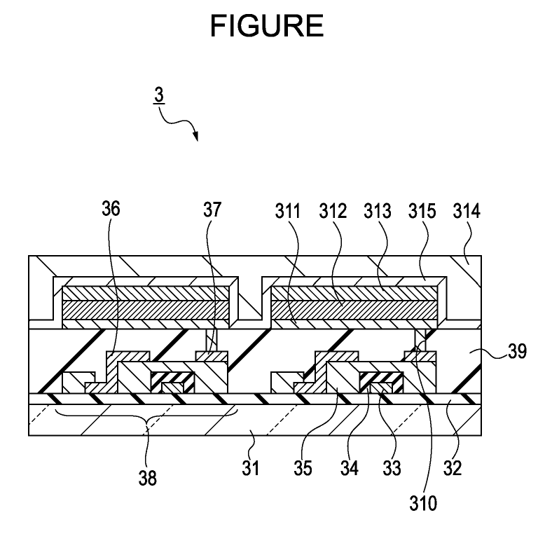

### TECHNICAL FIELD

The present invention relates to an organic light-emitting device including an emission layer containing two particular types of organic compounds.

### BACKGROUND ART

An organic light-emitting device includes an anode, a cathode, and a thin-film containing an organic compound and being disposed between the anode and the cathode. When electrons and holes are injected from the respective electrodes, excitons of a fluorescent compound are generated, and the organic light-emitting device emits light as these excitons return to their ground state.

Organic light-emitting devices may be incorporated in full color displays and the like. In such a case, emission of blue light with good color purity at high efficiency is necessary, but more improvements are desirable in this regard. Although attempts to use organic compounds having fluoranthene and benzofluoranthene skeletons have been made to address this need (refer to Patent Citations 1 to 4), still more improvements are desired.
Patent Citation 1
Japanese Patent Laid-Open No. 10-189247
Patent Citation 2
Japanese Patent Laid-Open No. 2005-235787
Patent Citation 3
WO2008-015945
Patent Citation 4
WO2008-059713

### DISCLOSURE OF INVENTION

It is desirable to provide an organic light-emitting device that exhibits emission hue with high purity, outputs high-luminance light at high efficiency, and has good durability.

An aspect of the present invention provides an organic light-emitting device that includes an anode, a cathode, and an emission layer disposed between the anode and the cathode, in which the emission layer contains a compound represented by general formula [1] below and a compound represented by general formula [2] below:

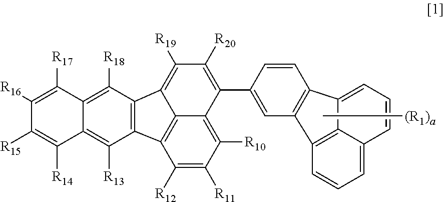

(in general formula [1], a represents an integer in the range of 0 to 9; R₁s each represent a group selected from a hydrogen atom, a halogen atom, a substituted or unsubstituted alkyl group, a substituted or unsubstituted aralkyl group, a substituted or unsubstituted aryl group, and a substituted or unsubstituted heterocyclic group and may be the same as or different from each other; and R₁₀ to R₂₀ each represent a group selected from a hydrogen atom, a halogen atom, a substituted or unsubstituted alkyl group, a substituted or unsubstituted aralkyl group, a substituted or unsubstituted aryl group, and a substituted or unsubstituted heterocyclic group and may be the same as or different from each other)

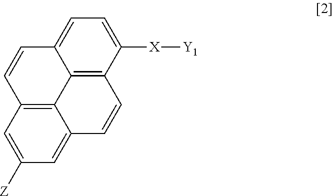

{in general formula [2], X represents a divalent substituted or unsubstituted fused ring aromatic group in which the number of fused rings is 2 or 3; Y₁ represents a group selected from a substituted or unsubstituted aryl group and a substituted or unsubstituted heterocyclic group; and Z represents a substituent represented by general formula [A] below:

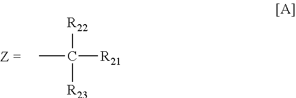

(in general formula [A], at least two of R₂₁ to R₂₃ represent substituted or unsubstituted alkyl groups and the remaining substituent is a hydrogen atom; and R₂₁ to R₂₃ may be the same as or different from each other.)}

Another aspect of the present invention provides a display apparatus comprising the organic light-emitting device described above and a unit for supplying electrical signals to the organic light-emitting device.

The present invention can provide an organic light-emitting device that exhibits emission hue with high purity, outputs high-luminance light at high efficiency, and has good durability.

### BRIEF DESCRIPTION OF DRAWINGS

FIGURE is a cross-sectional view of a display apparatus including an organic light-emitting device and a TFT for controlling luminance of emission from the organic light-emitting device.

### DESCRIPTION OF EMBODIMENTS

The present invention will now be described in details.

An organic light-emitting device according to an embodiment of the present invention includes an emission layer that contains two types of organic compounds having particular structures. Organic compounds having particular structures are organic compounds represented by general formulae [1] and [2] below.

A compound represented by general formula [1] used in the organic light-emitting element is first described. As shown below, the compound represented by general formula [1] has the 3-position of a benzo[k]fluoranthene ring bonded to the 8-position of a fluoranthene ring:

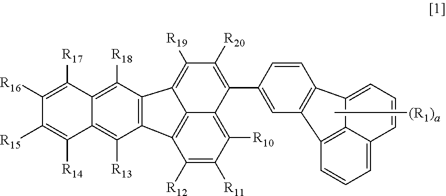

In general formula [1], a represents an integer in the range of 0 to 9; R₁s each represent a group selected from a hydrogen atom, a halogen atom, a substituted or unsubstituted alkyl group, a substituted or unsubstituted aralkyl group, a substituted or unsubstituted aryl group, and a substituted or unsubstituted heterocyclic group and may be the same as or different from each other; R₁₀ to R₂₀ each represent a group selected from a hydrogen atom, a halogen atom, a substituted or unsubstituted alkyl group, a substituted or unsubstituted aralkyl group, a substituted or unsubstituted aryl group, or a substituted or unsubstituted heterocyclic group and may be the same as or different from each other.

An anode and a cathode are provided as a pair of electrodes. At least one of the anode and the cathode is transparent or semitransparent (transmittance of about 50%) for the emission color.

The emission layer is the very layer that emits light. The organic light-emitting device may further include functional layers other than the emission layer. In such an organic light-emitting device, the emission layer is layered together with other functional layers. The layer structure of the organic light-emitting device is described below.

The emission layer contains the organic compound represented by general formula [1] above and an organic compound represented by general formula [2] below. How these organic compounds are distributed in the emission layer is described below. The emission layer may contain other components in addition to the compounds represented by general formulae [1] and [2].

In the emission layer, the organic compound represented by general formula [1] functions as a guest material. The organic compound represented by general formula [2] functions as a host material. For the purpose of this specification, “guest material” refers to a material that substantially defines the color of emission from the organic light-emitting device and is a material capable of emitting light. The host material is a material whose content is higher than the guest material content in the emission layer.

The guest material content is low and the host material content is high in the emission layer. Here, the “content” is in terms of percent by weight relative to all components that make up the emission layer.

The guest material content is 0.01 wt % or more and 80 wt % or less or can be 1 wt % or more and 40 wt % or less. These numerical ranges also apply when the emission layer is composed of a host material and a guest material only.

In the emission layer, the guest material may either be uniformly contained over all parts of the emission layer or may have a concentration gradient. Alternatively, the guest material may be contained in some parts of the emission layer but not in other parts.

As mentioned earlier, the emission layer of the organic light-emitting device may contain compounds other than the organic compounds represented by general formulae [1] and [2]. In such a case, the organic compound represented by general formula [2] does not have to be the compound whose content is the highest in the emission layer.

The host material has a larger energy gap than the guest material. The guest material receives excitation energy from the host material and emits light. The host/guest weight ratio in the emission layer is adjusted depending on the desired emission characteristics. To prevent concentration quenching, the guest can be dispersed in the host.

When the emission layer contains a host material and a guest material having a carrier transport property, the process leading to emission includes following steps:

1. Electrons and/or holes are transported in the emission layer;

2. The host material generates excitons;

3. Excitation energy is transferred among molecules of the host material; and

4. The excitation energy is transferred to the guest material from the host material.

The energy transfer in the respective steps and emission occur in competition with various deactivation steps. Naturally, in order to enhance the emission efficiency of the organic light-emitting device, the emission quantum yield of the emission center material itself must be high. How to efficiently transfer energy between the host molecules or between the host and the guest is also critical for the device design.

The exact cause for emission deterioration by electrical current is not yet clear. The inventors of the present invention believe that the emission center material (guest material) or environmental changes surrounding the emission center material (e.g., changes occurring in the guest material) may be attributable to emission deterioration by electrical current.

The inventors have conducted various investigations. As a result, the inventors have found that when an organic compound represented by general formula [1] above is used as the guest material, it is useful to use a host material having a particular structure in order to obtain an organic light-emitting device that emits blue light highly efficiently, maintains a high luminance over a long period of time, and undergoes less deterioration by electrical current. The host material having a particular structure is the material represented by general formula [2] described below.

In applying the organic light-emitting device to a pixel of a pixel unit of a display apparatus such as a flat panel display, the organic light-emitting device can be used as a blue light-emitting pixel in a display region of the display. The pixel at least includes the organic light-emitting device and a switching device for controlling the emission luminance, e.g., a TFT.

The compound represented by general formula [1] has an emission peak in the optimum range of 450 to 460 nm in a diluted solution since benzo[k]fluoranthene is bonded to fluoranthene.

In general, when organic light-emitting devices are used in displays, it is important that the emission peak of the blue light-emitting material contained in the blue light-emitting devices be in the range of 430 to 480 nm.

In other words, the organic light-emitting device of this embodiment not only contains a blue light-emitting material that has an emission peak within the range important for providing blue light-emitting devices. Rather, the organic light-emitting device of this embodiment contains a blue light-emitting material that has an emission peak within a range of 450 nm to 460 nm, which is narrower than the range of 430 nm to 480 nm.

The organic compound for the organic light-emitting device may have a molecular weight of 1000 or less. This is because sublimation purification can be employed as a purification technique. Sublimation purification is highly advantageous for increasing the purity of materials.

The organic compound contained in the organic compound layer of the organic light-emitting device, in particular, the organic compound represented by general formula [1], may have a molecular weight of 1000 or less.

The organic compound represented by general formula [1] contained in the organic compound layer of the organic light-emitting device is advantageous in that it has thermal stability. In forming an organic compound layer of an organic light-emitting device, the organic compound is subjected to steps such as sublimation purification and evaporation. In such a case, the organic compound is exposed to a temperature of 300° C. or higher in a high vacuum of about 10⁻³ Pa. If the thermal stability of the material is low, decomposition and reactions may occur and desired properties may not be obtained.

The organic compound represented by general formula [1] contained in the organic compound layer of the organic light-emitting device is stable against such heat. In contrast, organic compounds below undergo changes in structure due to heat.

For example, suppose that an organic compound that has a fluoranthene or benzo[k]fluoranthene skeleton is used but its 3-position is occupied by a substituent having a peri-position. In such an organic compound, the 4-position of fluoranthene or benzo[k]fluoranthene has a reactivity significantly higher than regular naphthalene and readily causes cyclization reaction by heat as shown below:

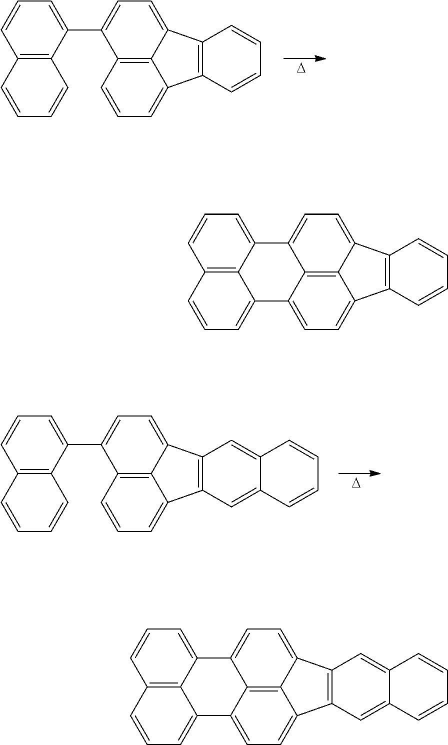

This implies that when such a material is used as the material for the organic light-emitting device, reaction may occur by heat applied during sublimation purification, evaporation, and operation. Once the cyclization reaction occurs, the absorption and emission wavelengths of the compound notably increase. In other words, the light emitted is in a wavelength range different from that of the original compound or the intensity of emission from the compound may decrease from the original level due to absorption by the cyclized compound.

This is particularly significant for molecular design of an organic compound having a fluoranthene or benzo[k]fluoranthene skeleton contained in the organic light-emitting device. The organic compound used in this embodiment has both a benzo[k]fluoranthene skeleton and a fluoranthene skeleton. However, since the 3-position of the benzo[k]fluoranthene ring is bonded to the 8-position of the fluoranthene ring, the organic compound does not have a position that can be cyclized by heat. Thus, chemical changes caused by heat applied during sublimation purification, evaporation, and operation can be suppressed.

The organic compound represented by general formula [1] contained in the emission layer of the organic light-emitting device may have phenyl groups respectively introduced to R₁₃ and R₁₈ at the center of the benzo[k]fluoranthene ring. This is because R₁₃ is sterically hindered by R₁₂ and R₁₄ and R₁₈ is sterically hindered by R₁₇ and R₁₈, and thus the molecular plane of the phenyl group becomes oriented substantially perpendicularly with respect to the benzo[k]fluoranthene ring plane. As a result, the concentration quenching and an increase in emission wavelength caused by interaction between fused heterocyclic groups of molecules can be suppressed. There is also an additional advantage that the wavelength of the emission from the molecules does not easily increase since the n-conjugated system of the benzo[k]fluoranthene skeleton does not expand to the perpendicularly oriented phenyl groups.

In general formula [1] above, at least one of R₁s, which are substituents on the fluoranthene ring, may be a substituted or unsubstituted alkyl group. The reason is as follows. Substituents on the fluoranthene ring are not easily sterically hindered by other substituents and hydrogen atoms compared to the benzo[k]fluoranthene ring. Thus, when aryl groups are introduced, the wavelength of emission tends to increase by the expansion of the π conjugated system or interactions between fused heterocyclic rings. Thus, in order to minimize changes in emission wavelength by these substituents, it is effective to introduce at least one substituted or unsubstituted alkyl group into the fluoranthene ring in the compound represented by general formula [1]. In this manner, the interactions with the fused heterocyclic groups of other molecules can be suppressed while maintaining the emission wavelength of the molecules at relatively the same level.

Specific examples of the substituents (R₁ and R₁₀ to R₂₀) in general formula [1] are as follows.

Examples of the halogen atom include fluorine, chlorine, bromine, and iodine atoms.

Examples of the substituted or unsubstituted alkyl group include, but are not limited to, a methyl group, an ethyl group, a normal propyl group, an iso-propyl group, a normal butyl group, a tert-butyl group, a sec-butyl group, an octyl group, a 1-adamantyl group, and a 2-adamantyl group.

Examples of the substituted or unsubstituted aralkyl group include, but are not limited to, a benzyl group.

Examples of the substituted or unsubstituted aryl group include a phenyl group, a naphthyl group, an indenyl group, a biphenyl group, a terphenyl group, and a fluorenyl group.

Examples of the substituted or unsubstituted heterocyclic group include, but are not limited to, a pyridyl group, an oxazolyl group, an oxadiazolyl group, a thiazolyl group, a thiadiazolyl group, a carbazolyl group, an acridinyl group, and a phenanthrolyl group.

Examples of the substituent that may be included in any one of the alkyl, aralkyl, aryl, heterocyclic groups include, but are not limited to, alkyl groups such as a methyl group, an ethyl group, and a propyl group; aralkyl groups such as a benzyl group; aryl groups such as a phenyl group and a biphenyl group; heterocyclic groups such as a pyridyl group and a pyrrolyl group; amino groups such as a dimethylamino group, a diethylamino group, a dibenzylamino group, a diphenylamino group, and a ditolylamino group; alkoxy groups such as a methoxyl group, an ethoxyl group, a propoxyl group, and a phenoxyl group; a cyano group; and halogen atoms such as fluorine, chlorine, bromine, and iodine atoms.

Specific examples of the compound represented by general formula [1] used in the present invention include, but are not limited to, those represented by the structural formulae below:

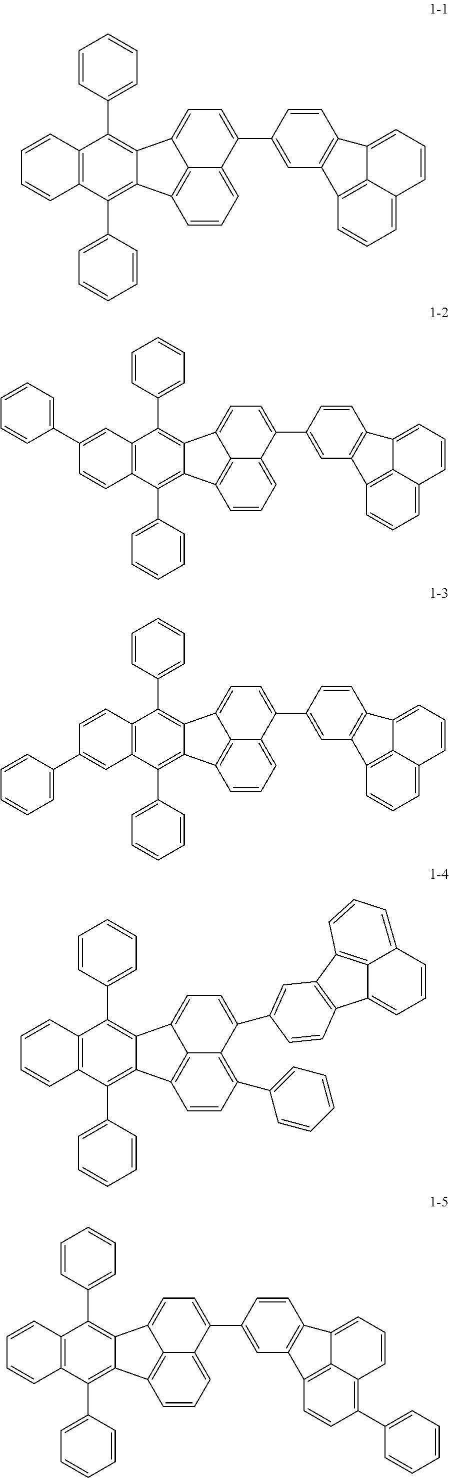

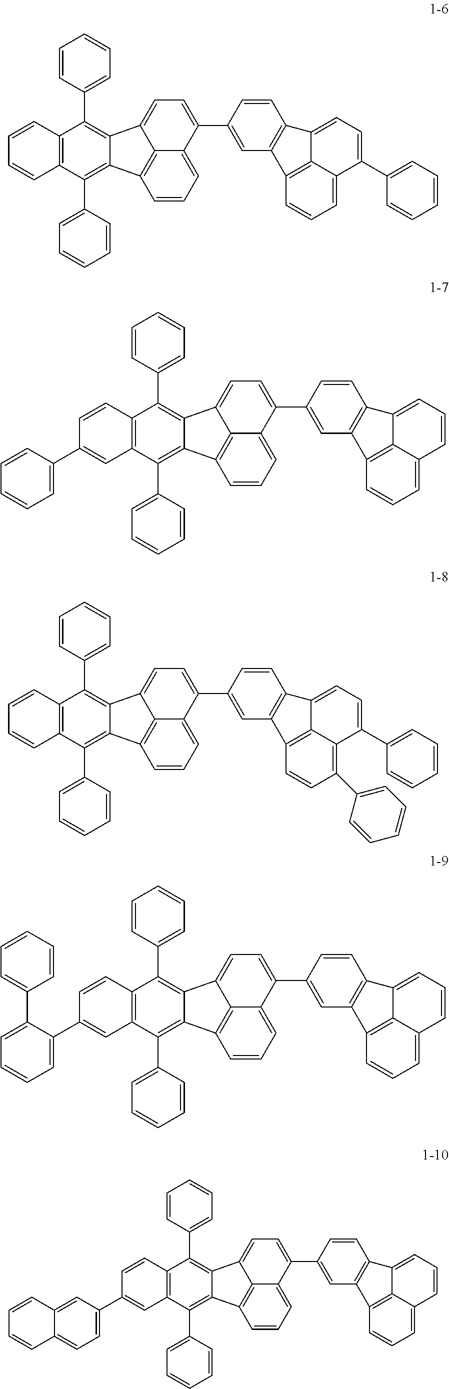

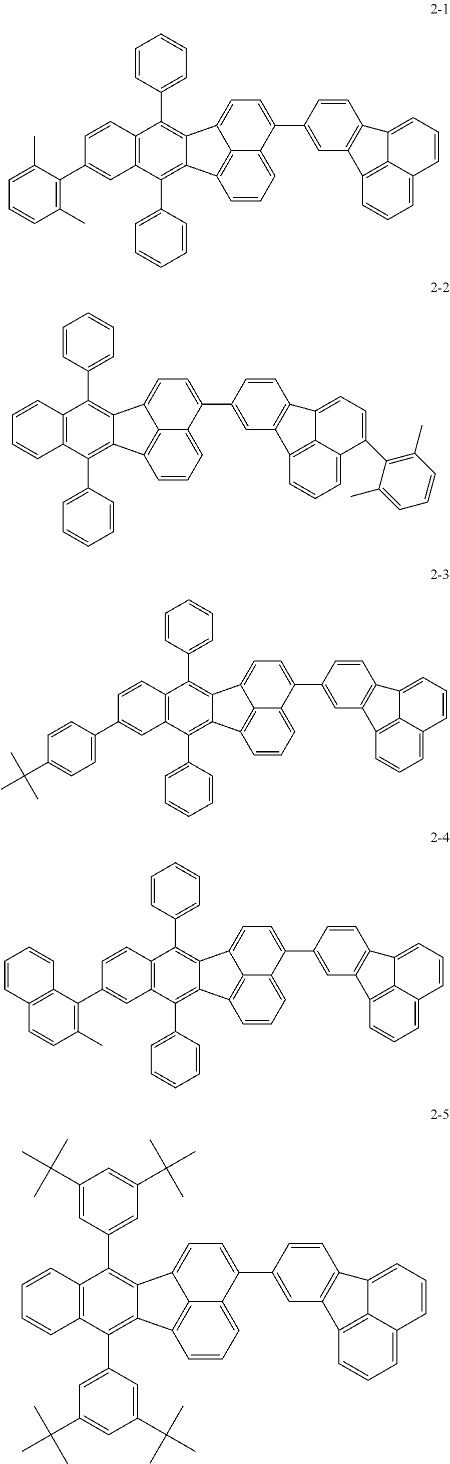

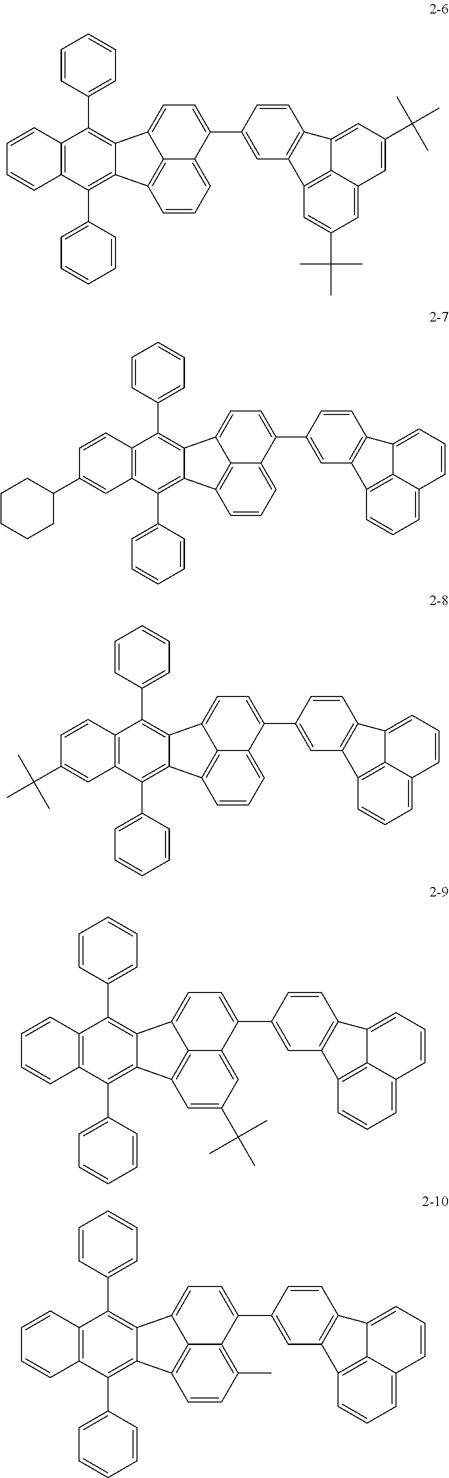

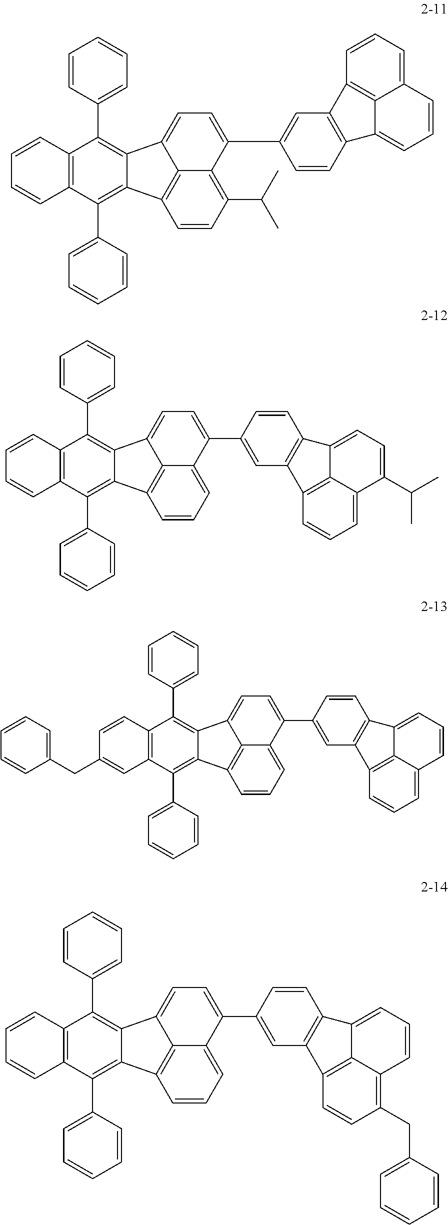

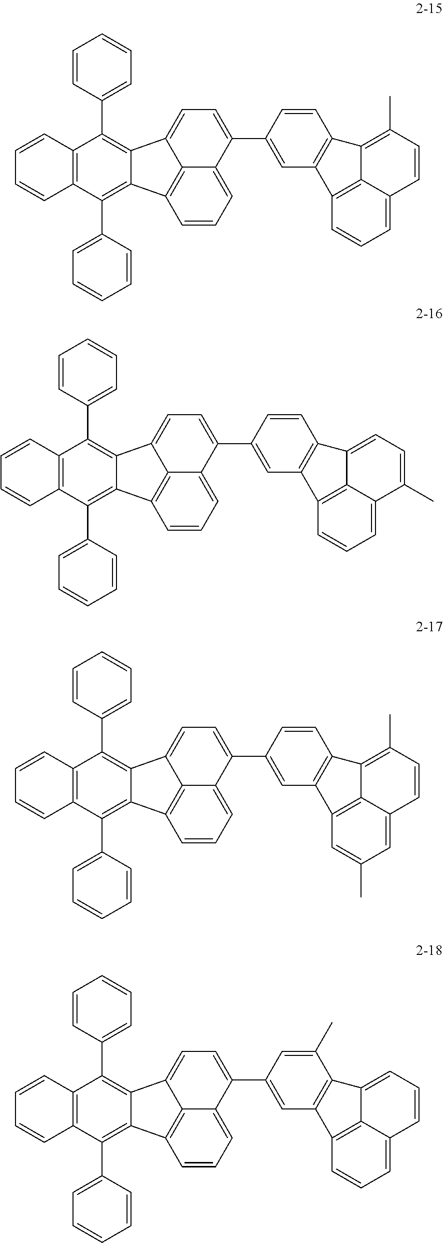

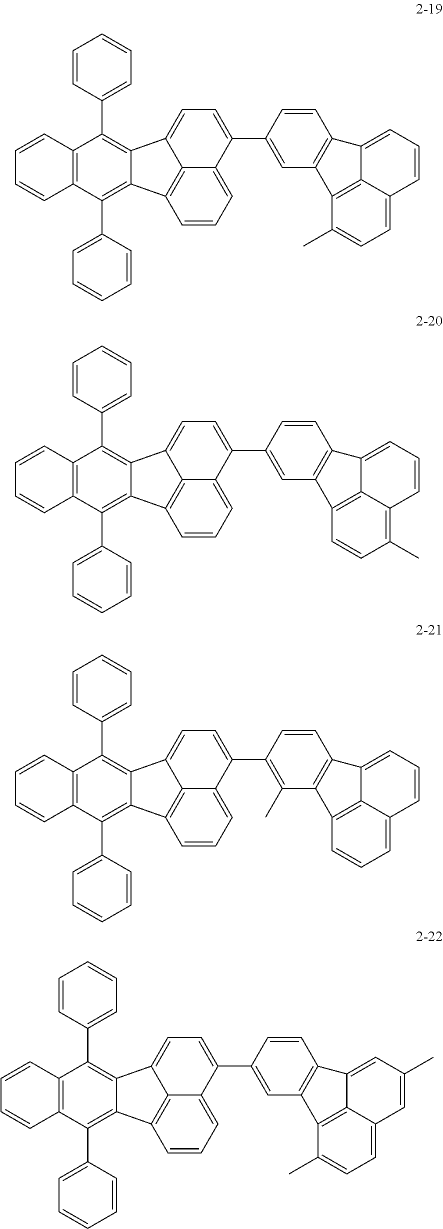

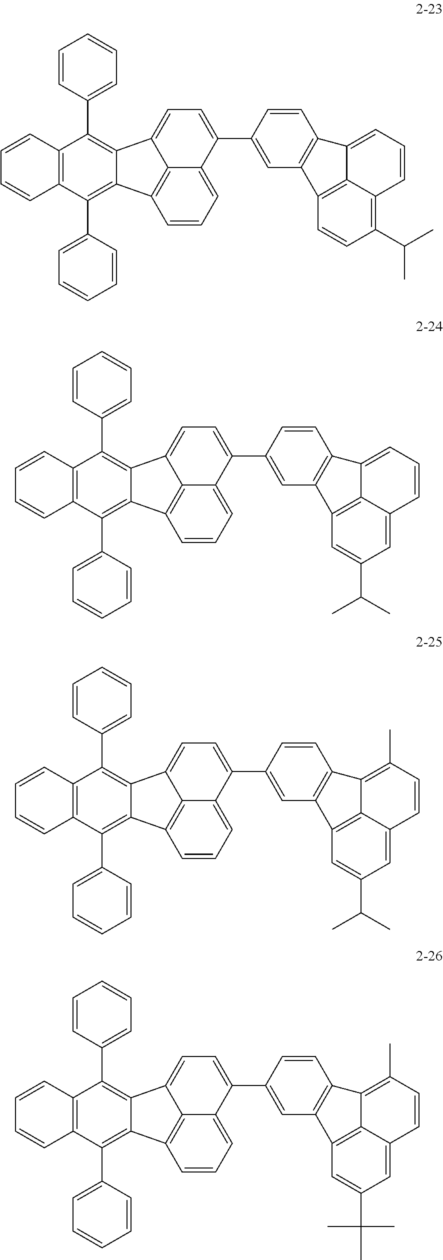

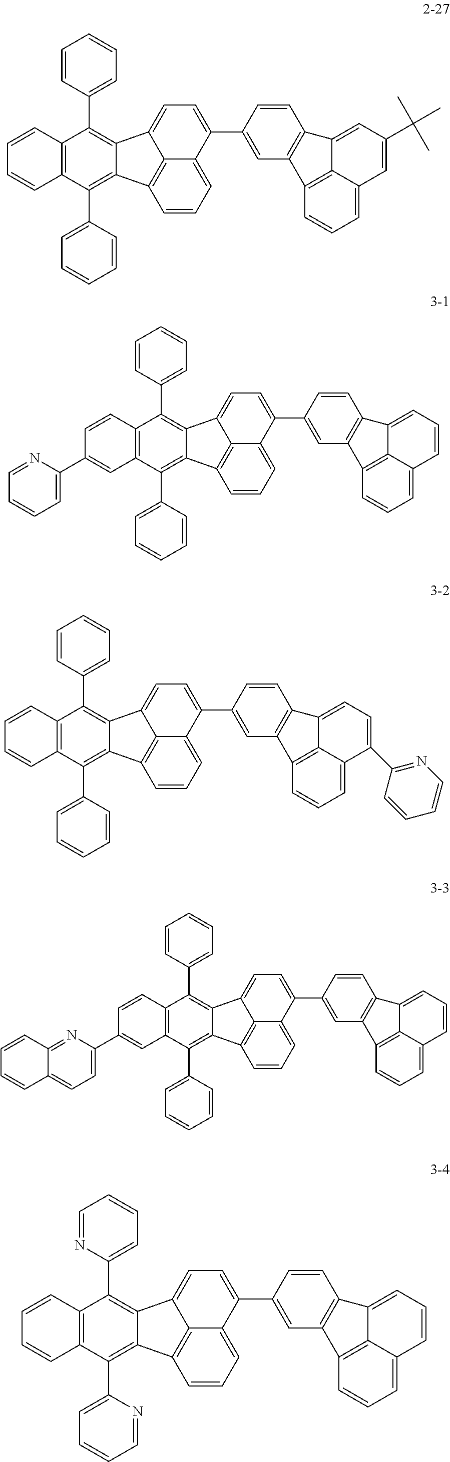

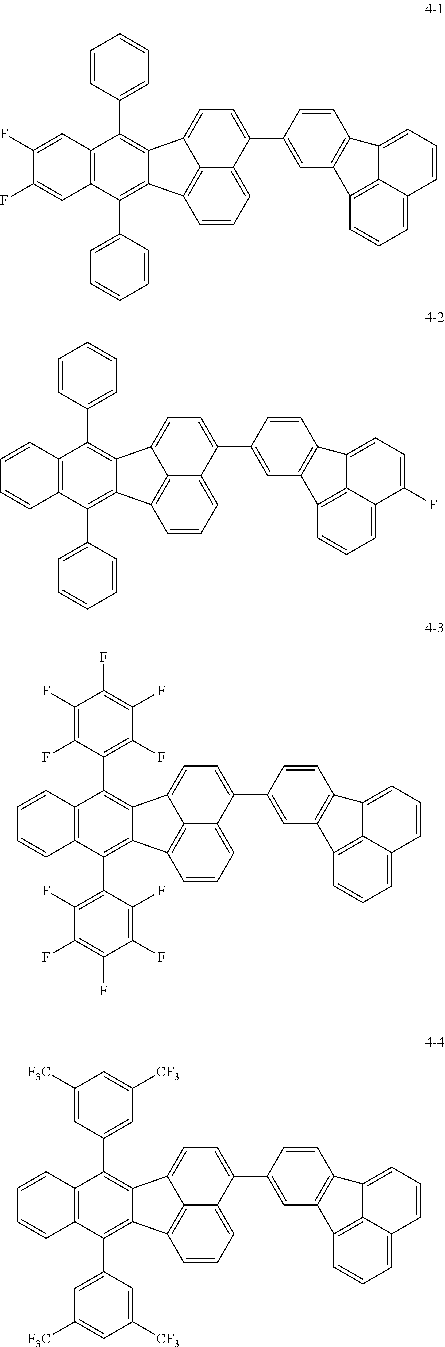

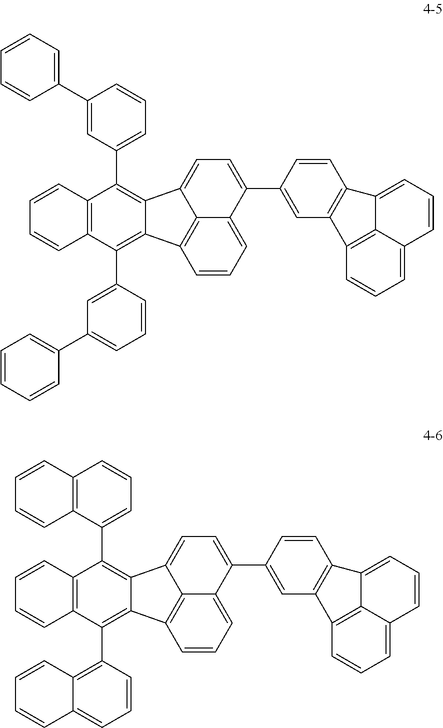

The compound represented by general formula [1] used in the present invention can be synthesized by a Suzuki-Miyaura coupling reaction between a corresponding brominated benzo[k]fluoranthene and a corresponding fluoranthene boronic acid pinacol ester, for example. The compound can also be synthesized by the reaction between a benzofluoranthene boronic acid pinacol ester and a halogenated or triflated fluoranthene. Boronic acid may be used instead of the boronic acid pinacol ester. Conversion to a boronic acid pinacol ester may be conducted through reacting a halogenated or triflated compound with 4,4,5,5-tetramethyl-[1,3,2]dioxaborolane in a toluene solvent in the presence of triethylamine and Ni(dppp)Cl₂ serving as a catalyst.

A non-limiting example of synthesis of 8-chlorofluoranthene is shown below:

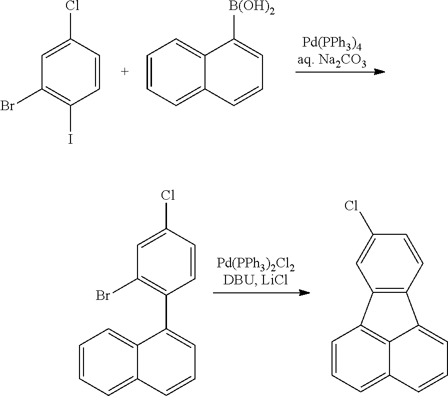

The tert-butyl-group-substituted fluoranthene unit can be synthesized by Friedel-Crafts alkylation of halogenofluoranthene.

The methods described above also apply to synthesis of compounds represented by general formula [2] described below.

A compound represented by general formula [2] will now be described.

The organic compound represented by general formula [2] contained in the emission layer of the organic light-emitting device of the present invention includes a pyrene skeleton including a fused ring aromatic group represented by X at the 1-position and a secondary or tertiary alkyl group represented by Z at the 7-position:

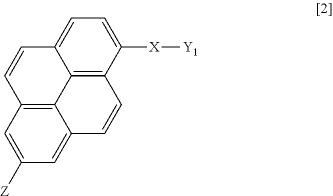

In general formula [2], X represents a divalent substituted or unsubstituted fused ring aromatic group in which the number of fused rings is 2 or 3; Y₁ represents a group selected from substituted or unsubstituted aryl groups and substituted or unsubstituted heterocyclic groups; and Z represents a substituent represented by general formula [A] below:

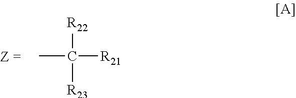

In general formula [A], at least two of R₂₁ to R₂₃ are substituted or unsubstituted alkyl groups and the remaining substituent is a hydrogen atom, and R₂₁ to R₂₃ may be the same as or different from each other.

The compound represented by general formula [2] has an energy gap larger than that of the compound represented by general formula [1]. In general formula [2], when the number of fused rings in X, i.e., a divalent fused ring aromatic group, bonded to pyrene is 4 or more, the energy gap of the compound represented by general formula [2] becomes close to or smaller than the energy gap of the guest. This is not desirable since the energy transfer efficiency between the host and the guest decreases.

When the energy gap of the host material is excessively large, an energy barrier is generated between the host material and the HOMO or LUMO level of the adjacent organic layer. This is also not desirable since the voltage of the organic light-emitting device increases.

For the reasons described above, the number of fused rings in X in general formula [2] may be 2 or 3. In such a case, the divalent substituted or unsubstituted fused ring aromatic group represented by X in general formula [2] may be any one of, but is not limited to, a naphthylene group, a fluorenylene group, an anthrylene group, and a phenanthrylene group.

Specific examples of a substituted or unsubstituted aryl group or a substituted or unsubstituted heterocyclic group represented by Y₁ in general formula [2] include those described in relation to general formula [1] above.

In a fused ring aromatic group having a wide n conjugate plane such as a pyrene skeleton, stacking of ring planes readily occurs, and the wavelength of the fluorescence increases and the excimer emission readily occurs by interactions between n electrons when an organic film is formed. When a pyrene-skeleton-containing compound is used as the host material, lowering of the energy gap is not desirable since it decreases the energy transfer efficiency between the host and the guest.

In addressing this problem, the inventors have found that ring plane stacking can be avoided by introducing into the pyrene skeleton an alkyl group having a small interaction with the fused ring aromatic group. In other words, as with Z in general formula [2], it is effective to introduce a bulky secondary or tertiary alkyl group (represented by general formula [A]) to the 7-position of the pyrene skeleton. The 7-position is where the alkyl group can be easily introduced by synthesis.

Specific examples of the substituted or unsubstituted alkyl group in general formula [A] are the same as those described in relation to the compound represented by general formula [1].

Examples of the compound represented by general formula [2] include, but are not limited to, the following:

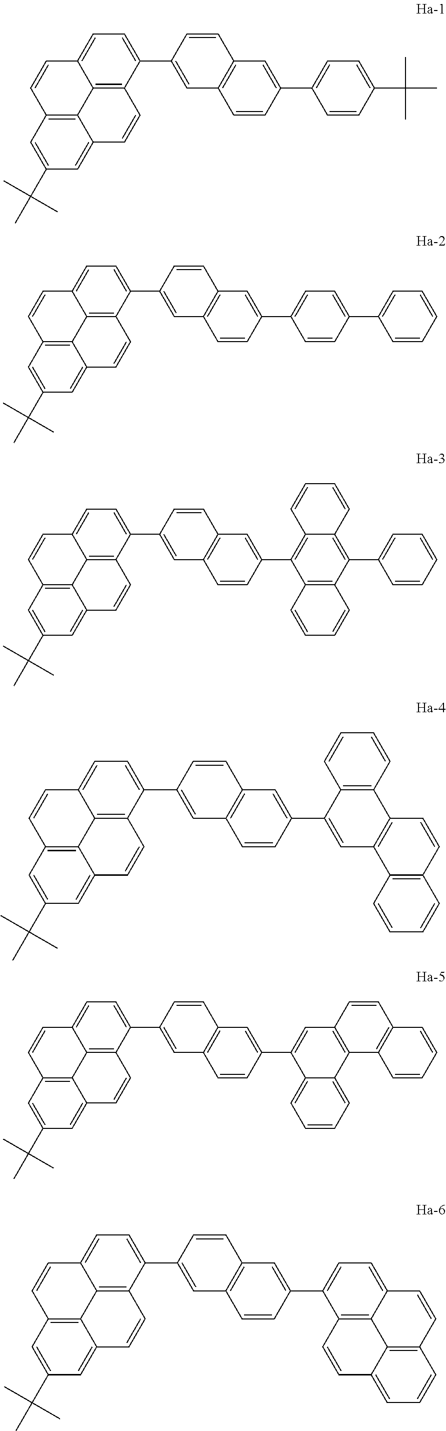

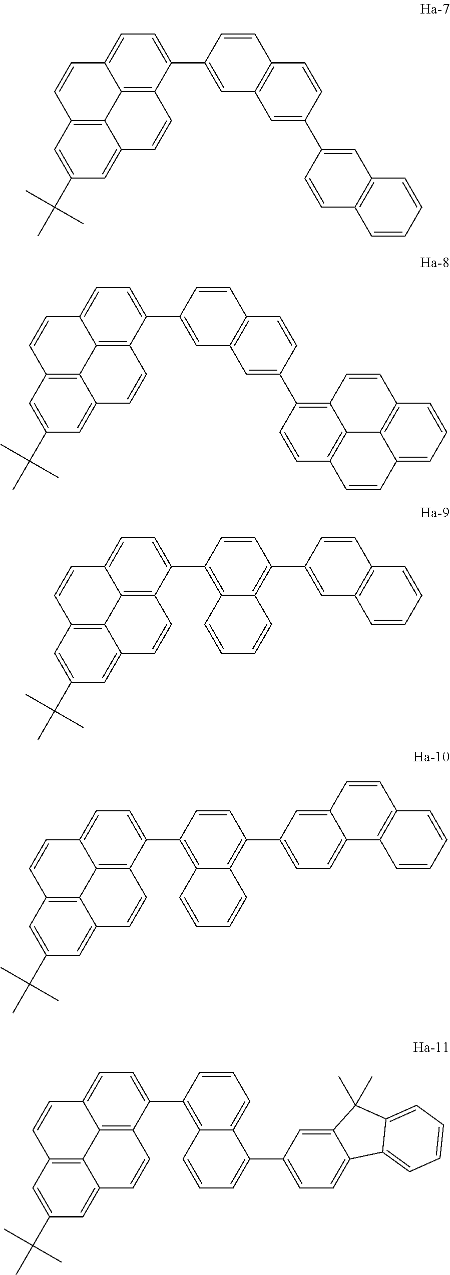

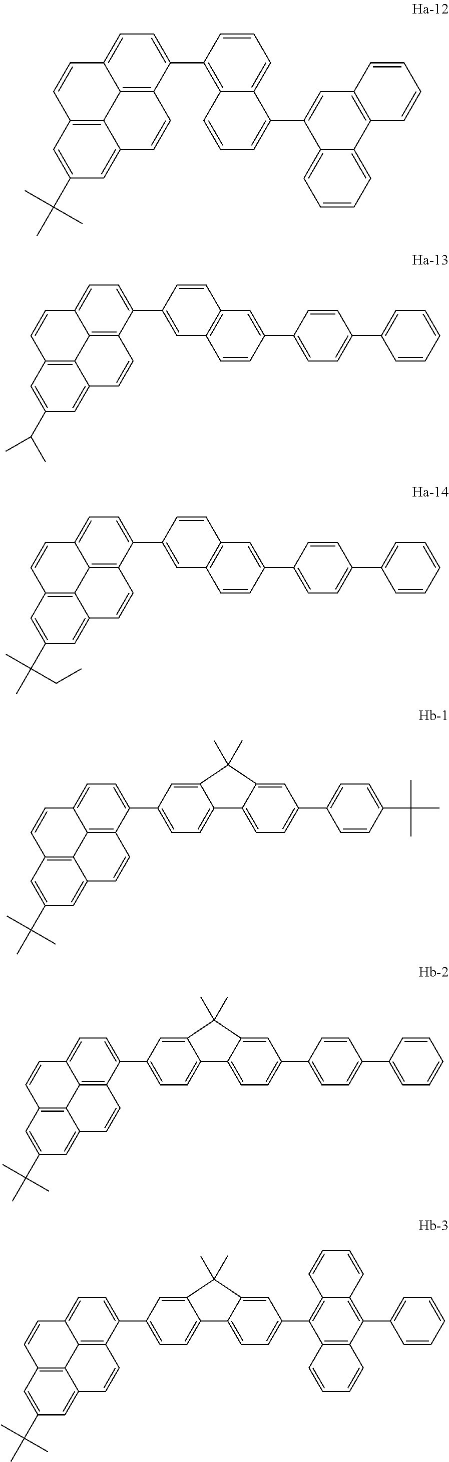

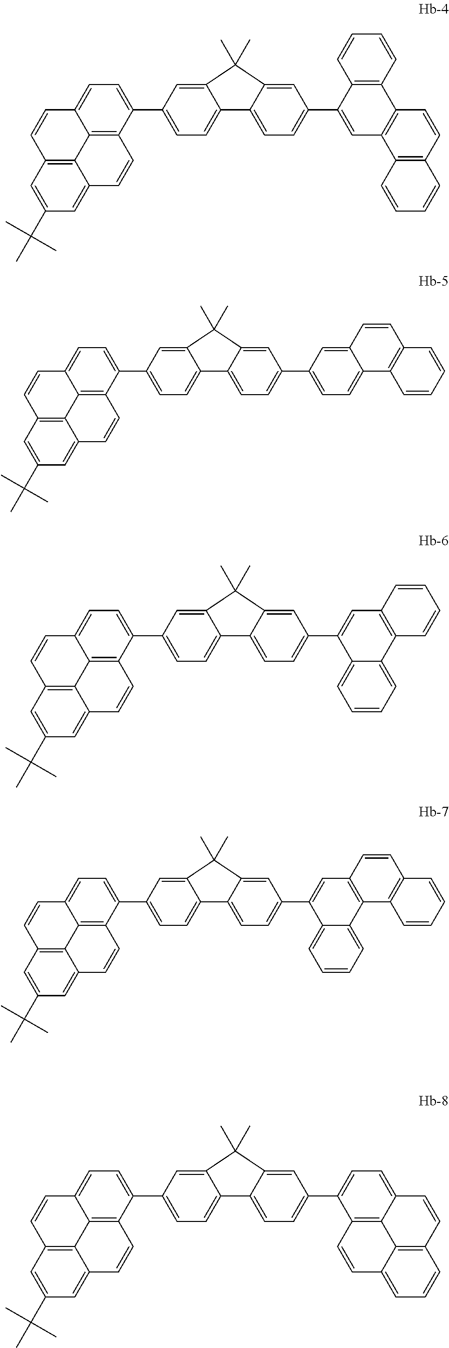

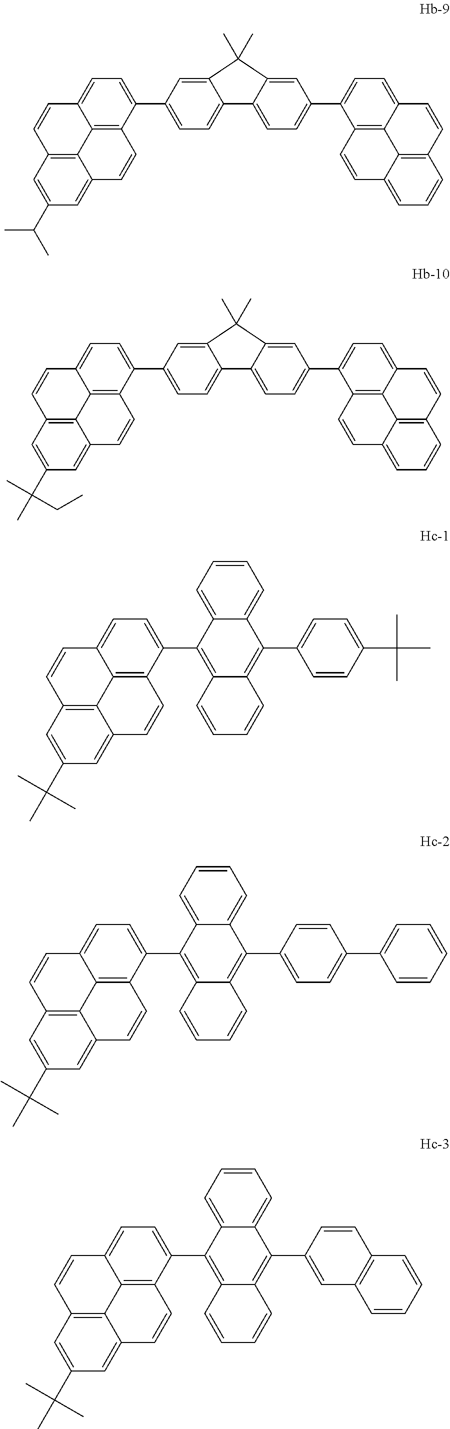

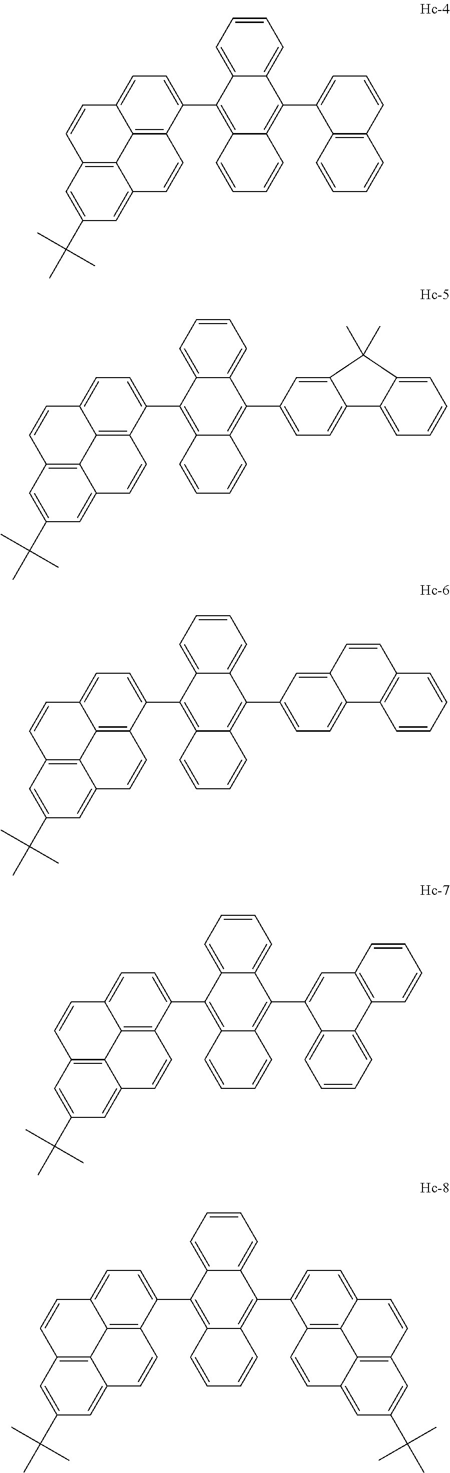

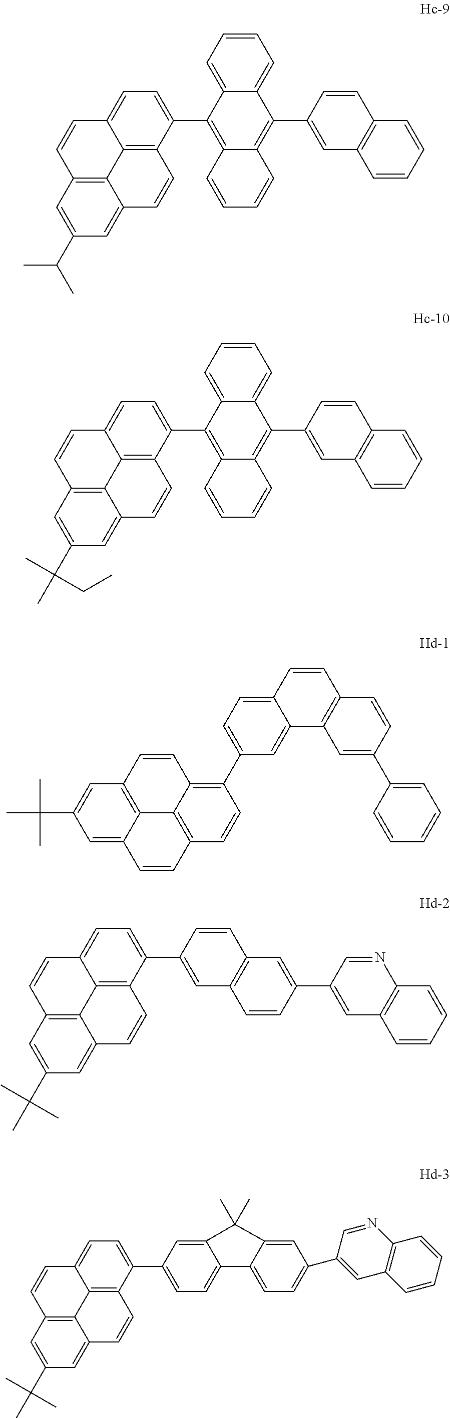

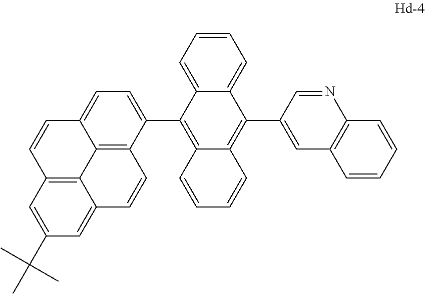

When a compound represented by general formula [3] or [4] is used as the compound represented by general formula [2], good emission that achieves a high emission efficiency and little luminance deterioration can be obtained continuously.

The amorphous property, the carrier mobility, and the energy gap of organic films can be adjusted by introduction of a variety of fused ring aromatic substituents into the Y₂ and Y₃ positions in general formulae [3] and [4] below. The studies conducted by the inventors have revealed that Y₂ may be a 2-naphthyl group, a 2-(9,9-dimethyl)fluorenyl group, a 2-phenanthryl group, a 9-phenanthryl group, or a 1-(7-tert-butyl)pyrenyl group, and that Y₃ may be a 2-naphthyl group, a 2-(9,9-dimethyl)fluorenyl group, or a 1-(7-tert-butyl)pyrenyl group.

As shown in Table 1, the energy gap of each of Example compounds He-1, He-2, He-3, He-4, He-5, Hf-1, Hf-2, and Hf-3 represented by general formulae [3] or [4] is about 3 eV. Since the energy gap of Example Compound 1-1 represented by general formula [1] used as the guest material is 2.78 eV, compounds represented by general formulae [3] and [4] can serve as the host material that achieves efficient energy transfer:

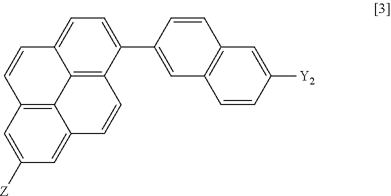

(in general formula [3], Y₂ represents a group selected from the group consisting of a 2-naphthyl group, a 2-(9,9-dimethyl)fluorenyl group, a 2-phenanthryl group, a 9-phenanthryl group, and a 1-(7-tert-butyl)pyrenyl group.),

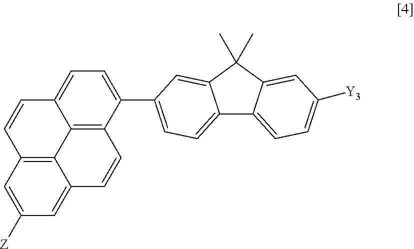

(in general formula [4], Y₃ represents a group selected from the group consisting of a 2-naphthyl group, a 2-(9,9-dimethyl)fluorenyl group, and a 1-(7-tert-butyl)pyrenyl group.)

When the compound represented by general formula [2] contained in the organic compound layer included in the organic light-emitting device of the present invention is a compound represented by general formula [3] or [4], the organic compound represented by general formula [1] may be as follows.

That is, in the organic compound represented by general formula [1], a is an integer in the range of 0 to 9, R₁s may each represent a group selected from a hydrogen atom, a halogen atom, a substituted or unsubstituted alkyl group, a substituted or unsubstituted aralkyl group, a substituted or unsubstituted aryl group, and a substituted or unsubstituted heterocyclic group, and may be the same as or different from each other.

However, when a is 1 or more, one or more R₁s may be substituted or unsubstituted alkyl groups which are the same as or different from each other.

Among R₁₀ to R₂₀, R₁₃ and R₁₈ may each represent an unsubstituted phenyl group, and R₁₀ to R₁₂, R₁₄ to R₁₇, R₁₉, and R₂₀ may each represent a group selected from a hydrogen atom, a halogen atom, a substituted or unsubstituted alkyl group, a substituted or unsubstituted aralkyl group, a substituted or unsubstituted aryl group, and a substituted or unsubstituted heterocyclic group. These groups may be the same as or different from each other.

Examples of the compound represented by general formulae [3] and [4] include, but are not limited to, the following:

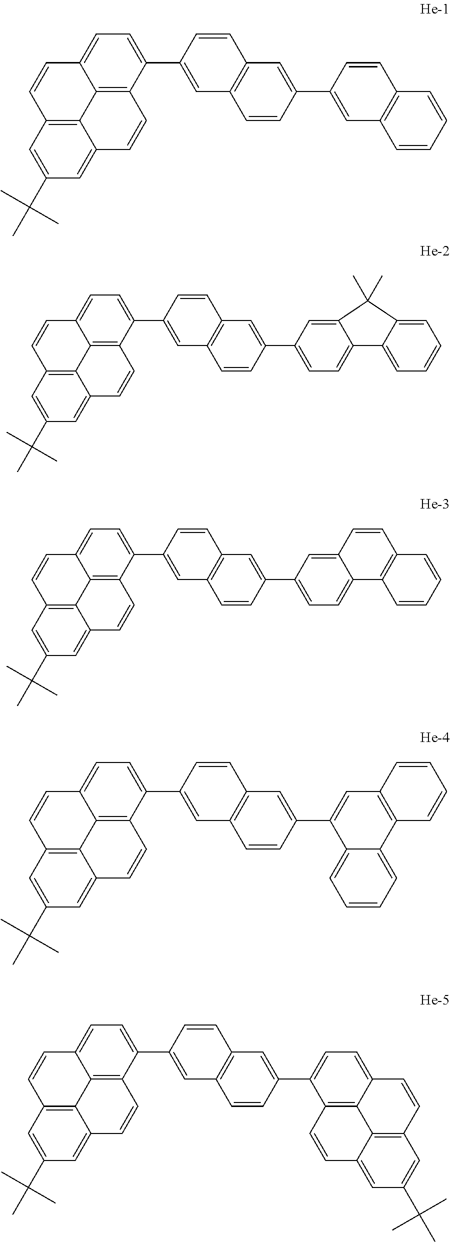

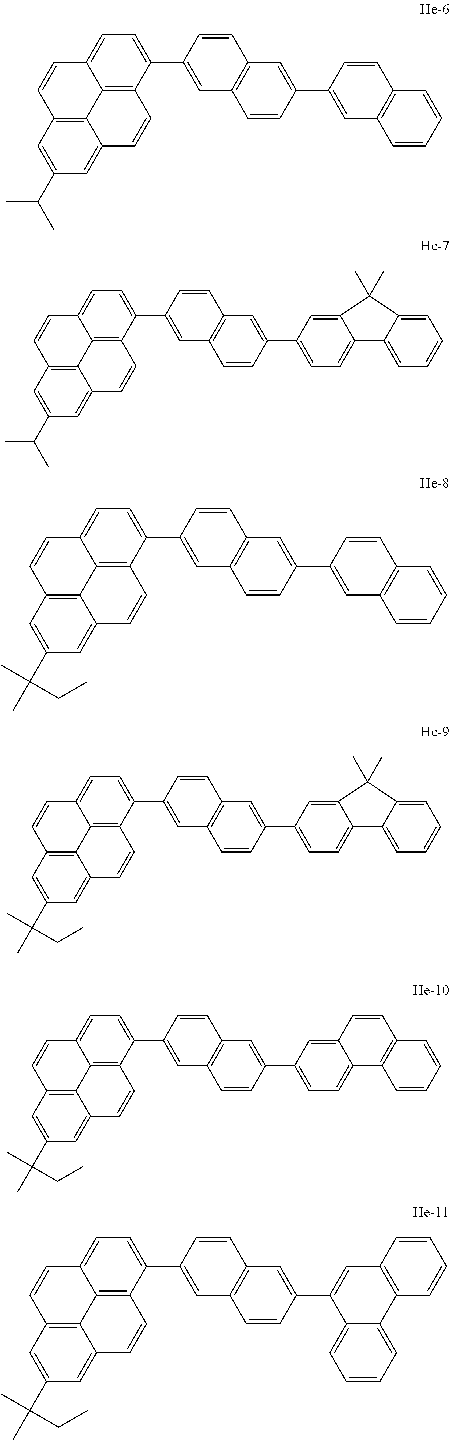

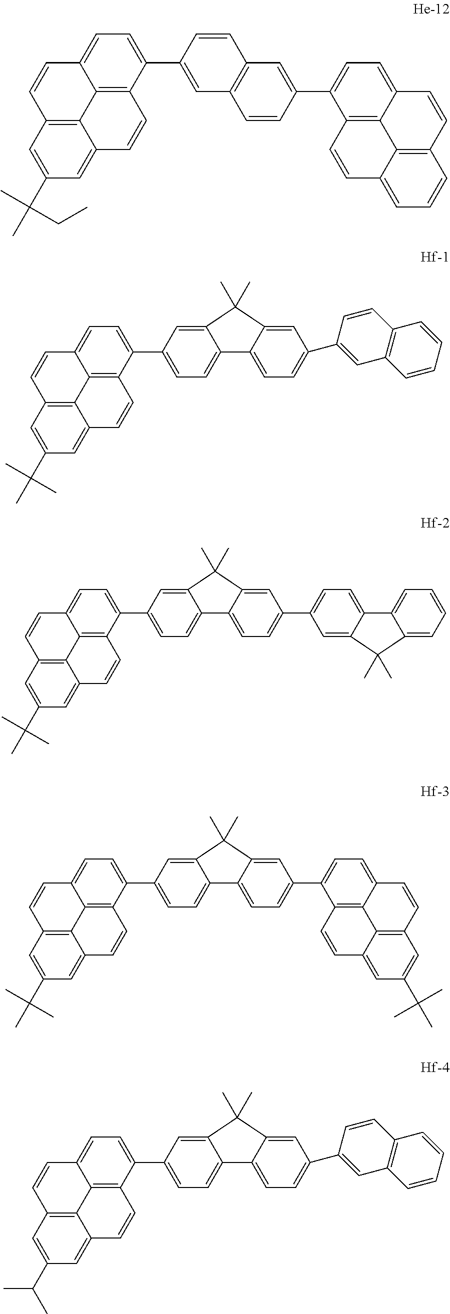

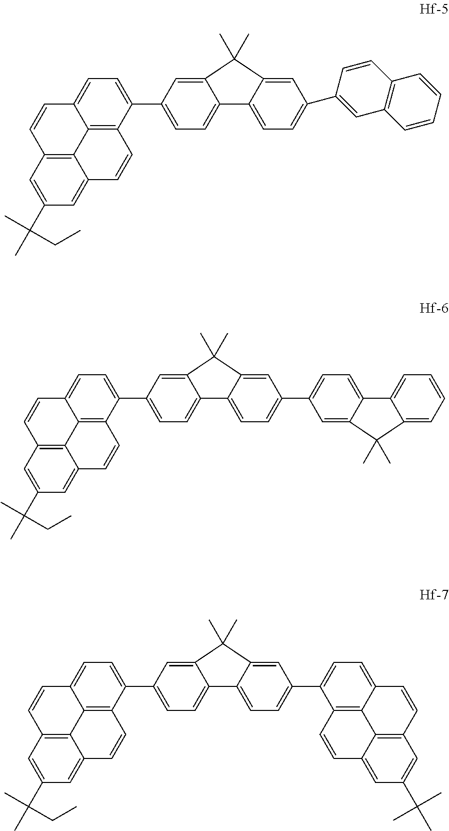

First to fifth examples of multilayered organic light-emitting devices are described below.

A first example of a structure of a multilayered organic light-emitting device is a structure in which an anode, an emission layer, and a cathode are sequentially layered on a substrate.

A second example of a structure of a multilayered organic light-emitting device is a structure in which an anode, a hole transport layer, an electron transport layer, and a cathode are sequentially layered on a substrate. In this case, the emission layer containing the guest material may be either one of the hole transport layer and the electron transport layer.

A third example of a structure of a multilayered organic light-emitting device is a structure in which an anode, a hole transport layer, an emission layer, an electron transport layer, and a cathode are sequentially layered on a substrate.

A fourth example of a structure of a multilayered organic light-emitting device is a structure in which an anode, a hole injection layer, a hole transport layer, an emission layer, an electron transport layer, and a cathode are sequentially layered on a substrate.

A fifth example of a structure of a multilayered organic light-emitting device is a structure in which an anode, a hole transport layer, an emission layer, a hole/exciton-blocking layer, an electron transport layer, and a cathode are sequentially layered on a substrate.

The multilayer structures of the first to fifth examples are only the basic device structures and do not limit the structure of the organic light-emitting element. For example, various other layer structures can be employed such as providing an insulating layer at the interface between an electrode and an organic layer, providing an adhesive layer or an optical interference layer, designing the hole transport layer to be made up of two layers with different ionization potentials, etc.

The emission layer of the organic light-emitting device may contain a hole transport compound, a light-emitting compound, or an electron transport compound of a low-molecular-weight-type or a polymer type in addition to the organic compounds represented by general formulae [1] and [2].

Examples of such compounds are as follows.

Examples of the hole injection and transport materials used for the hole injection layer and the hole transport layer include low-molecular-weight materials such as triarylamine derivatives, phenylenediamine derivatives, triazole derivatives, oxadiazole derivatives, imidazole derivatives, pyrazoline derivatives, pyrazolone derivatives, oxazole derivatives, fluorenone derivatives, hydrazone derivatives, stilbene derivatives, phthalocyanine derivatives, and porphyrin derivatives; and poly(vinyl carbazole), polysilylene, polythiophene, and other conductive polymers.

Examples of the electron injection and transport materials used for the electron injection and transport layers include oxadiazole derivatives, oxazole derivatives, thiazole derivatives, thiadiazole derivatives, pyrazine derivatives, triazole derivatives, triazine derivatives, perylene derivatives, quinoline derivatives, quinoxaline derivatives, fluorenone derivatives, anthrone derivatives, phenanthroline derivatives, and organometallic complexes.

In the organic light-emitting device of the present invention, the emission layer containing the compounds represented by general formulae [1] and [2] and layers composed of other organic compounds can be formed by the following thin-film-forming techniques: vacuum vapor deposition, ionization deposition, sputtering, plasma-enhanced deposition, and application processes that use solutions of materials in adequate solvents (e.g., spin-coating, dipping, casting, Langmuir-Blodgett (LB), and ink-jet techniques).

When layers are formed by vacuum vapor deposition or a solution application technique, crystallization and other unfavorable phenomena rarely occur and stability with time is excellent. When an application technique is used to form films, an adequate binding resin may be used in combination.

The binding resin may be selected from a wide range of binding resins. Examples of the binding resin include, but are not limited to, polyvinyl carbazole resins, polycarbonate resins, polyester resins, polyarylate resins, polystyrene resins, ABS resins, polybutadiene resins, polyurethane resins, acryl resins, methacryl resins, butyral resins, polyvinyl acetal resins, polyamide resins, polyimide resins, polyethylene resins, polyethersulfone resins, diallyl phthalate resins, phenol resins, epoxy resins, silicone resins, polysulfone resins, and urea resins.

These binding resins may be used alone or in combination as a copolymer. If needed, additives such as a plasticizer, an antioxidant, and a UV absorber may be used in combination.

The material for the anode may be a material that has a large work function. Examples thereof include single metals such as gold, platinum, silver, copper, nickel, palladium, cobalt, selenium, vanadium, and tungsten, and their alloys; and metal oxides such as tin oxide, zinc oxide, indium oxide, indium tin oxide (ITO), and zinc indium oxide. Electrically conductive polymers such as polyaniline, polypyrrole, polythiophene, and polyphenylene sulfide can also be used. These electrode substances may be used alone or in combination. The anode may have a single-layer structure or a multilayer structure.

In contrast, the material for the cathode may be a material that has a small work function. Examples of the cathode material include single metals such as lithium, sodium, potassium, cesium, calcium, magnesium, aluminum, indium, ruthenium, titanium, manganese, yttrium, silver, lead, tin, and chromium. Alloys of two or more metals such as lithium-indium, sodium-potassium, magnesium-silver, aluminum-lithium, aluminum-magnesium, and magnesium-indium, may also be used. Metal oxides such as indium tin oxide (ITO) may also be used. These electrode substances may be used alone or in combination. The cathode may have a single-layer structure or a multilayer structure.

The substrate used in the organic light-emitting device of the present invention is not particularly limited. For example, an opaque substrate such as a metal substrate or a ceramic substrate or a transparent substrate such as a glass substrate, a quartz substrate, or a plastic sheet, may be used. A color filter film, a fluorescence color conversion filter film, a dielectric reflective film, or the like may be formed on the substrate to control the color of emission.

A protective layer and/or a sealing layer may be provided to the fabricated device in order to prevent the device from contacting oxygen, moisture, and the like.

Examples of the protective layer include inorganic material films such as diamond thin films and metal oxide and metal nitride films; polymeric films of fluorocarbon resin, polyethylene, silicone resin, and polystyrene resin; and films of photocurable resin. The device may be covered with glass, a gas-impermeable film, a metal, or the like and packaged with an adequate sealing resin.

As for the direction in which the light is output from the device, a bottom-emission structure (light is output from the substrate side) or a top-emission structure (light is output from the side opposite to the substrate) may be employed.

The organic light-emitting device of the present invention can be applied to products that require energy saving and high luminance. Examples of the application include display apparatuses (flat panel displays), lighting apparatuses, light sources for printers, and backlights for liquid crystal displays.

When the organic light-emitting device is used in a display apparatus, a color filter film, a fluorescence color conversion filter film, a dielectric reflective film, and other associated components may be formed on the substrate to control the color of emission.

FIGURE is a schematic diagram showing an example of a cross-sectional structure of a TFT, an organic light-emitting device, and a substrate that constitute a display apparatus 3. A moisture-proof film 32 is disposed on a surface of a substrate 31, such as a glass substrate, to protect components (TFT or organic layer) formed thereon. Silicon oxide, a complex of silicon oxide and silicon nitride, or the like is used to form the moisture-proof film 32. A gate electrode 33 is disposed on the moisture-proof film 32. The gate electrode 33 is obtained by depositing a metal such as chromium by sputtering.

A gate insulating film 34 is provided to cover the gate electrode 33. The gate insulating film 34 is obtained by depositing silicon oxide or the like by a plasma-enhanced chemical vapor deposition (CVD) method or a catalytic chemical vapor deposition (cat-CVD) method and patterning the resulting film.

A semiconductor layer 35 is disposed to cover the gate insulating film 34. The semiconductor layer 35 is obtained by forming a silicon film by plasma-enhanced CVD or the like (if necessary, annealing at 290° C. or higher may be performed) and patterning the silicon film according to a circuit shape.

A TFT device 38 includes the gate electrode 33, the gate insulating film 34, the semiconductor layer 35, a drain electrode 36, and a source electrode 37. The drain electrode 36 and the source electrode 37 are distant from each other and are disposed on the semiconductor layer 35. In the drawing, two TFT devices 38 are disposed in the same plane. An insulating film 39 is disposed to cover the TFT devices 38. A contact hole (through hole) 310 composed of a metal is arranged to connect the source electrode 37 of the TFT device 38 to an anode 311 for the organic light-emitting device.

A multilayer or single-layer organic layer 312 and a cathode 313 are sequentially layered on the anode 311. They constitute the organic light-emitting device.

The display apparatus 3 includes the organic light-emitting device and the TFT devices 38. As shown in the drawing, a first protective layer 314 and a second protective layer 315 may be provided to prevent deterioration of the organic light-emitting device.

Note that the switching device of the display apparatus described above is not particularly limited, and the display apparatus can easily be applied even with a single crystal silicon substrate, a MIM device, an a-Si device, or the like.

An organic light-emitting display panel can be obtained by sequentially layering a single-layer or multilayer organic emission layer and a cathode layer on the ITO electrode. When the display panel containing the organic compounds of the present invention is driven, high-quality images can be displayed stably over a long time.

As for the direction in which the light is output from the device, a bottom-emission structure (light is output from the substrate side) or a top-emission structure (light is output from the side opposite the substrate) may be employed.

### EXAMPLES

Examples will now be described. The present invention is not limited by these examples.

### Production Example 1

### [Method for Making Example Compound 1-1]

Example Compound 1-1, which is one example of the organic compound represented by general formula [1] contained in the organic compound layer of the organic light-emitting device of the present invention, can be made by the following method, for example:

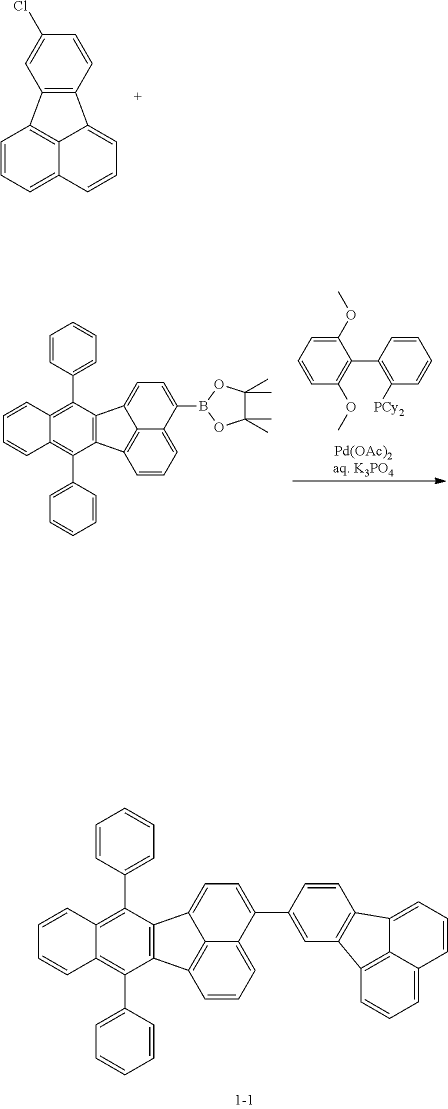

In a nitrogen atmosphere, compounds below were dissolved in a mixed solvent of toluene (30 ml) and ethanol (5 ml), an aqueous solution of 0.896 g (4.22 mmol) of potassium phosphate dissolved in 5 ml of distilled water was added to the resulting solution, and the resulting mixture was stirred for 12 hours in a silicone oil bath heated to 90° C. under heating: 8-chlorofluoranthene 0.50 g (2.11 mmol), 2-(7,12-diphenylbenzo[k]fluoranthen-3-yl)-4,4,5,5-tetramethyl-1,3,2-dioxaborolane 1.23 g (2.32 mmol), 2-dicyclohexylphosphino-2′,6′-dimethoxybiphenyl 0.0953 g (0.232 mmol), and palladium acetate 0.0237 g (0.106 mmol).

The resulting mixture was cooled to room temperature; water, toluene, and ethyl acetate were added thereto; and the organic layer was separated. The water layer was extracted (twice) with a mixed solvent of toluene and ethyl acetate and added to the solution of the organic layer separated initially. The organic layer was washed with saturated saline and dried with sodium sulfate. The solvent was distilled off and the residue was purified by silica gel column chromatography (toluene/heptane (1:3) mobile phase). The purified residue was vacuum dried at 120° C. and subjected to sublimation purification. As a result, 0.829 g (yield: 65%) of Example Compound 1-1 was obtained as a pale yellow solid.

Matrix-assisted laser desorption ionization-time of flight mass spectrometry (MALDI-TOF MS) was conducted to confirm 604.7, which was M⁺ of the compound.

The structure of the compound was confirmed by ¹H-NMR spectroscopy.

¹H-NMR (CDCl₃, 400 MHz) δ (ppm): 8.05 (1H, d, J=0.92 Hz), 8.00 (1H, d, J=7.67 Hz), 7.98 (1H, d, J=3.55 Hz), 7.93 (1H, d, J=3.55 Hz), 7.91 (1H, d, J=7.67 Hz), 7.87 (2H, d, J=8.24 Hz), 7.73-7.59 (13H, m), 7.53 (1H, dd, J=7.79, 1.60 Hz), 7.45-7.40 (3H, m), 7.36-7.32 (2H, m), 6.71 (1H, d, J=7.33 Hz), 6.67 (1H, d, J=7.10 Hz)

A photoluminescence (PL) spectrum of a 1×10⁻⁵ mol/l toluene solution containing Example Compound 1-1 was measured with F-4500 produced by Hitachi Ltd., at an excitation wavelength of 350 nm. A blue emission spectrum having the maximum intensity at 449 nm was detected.

The absorption spectrum of the toluene solution was measured with V-560 produced by JASCO Corporation to calculate the energy gap of Example Compound 1-1. The energy gap of Example Compound 1-1 was 2.78 eV.

Note that the energy gap was calculated as the energy of the wavelength at the point of intersection between the wavelength axis and the tangent line drawn to the long wavelength-side absorption edge of the absorption spectrum.

### Production Example 2

### [Method for Making Example Compound 2-5]

Example Compound 2-5 was synthesized as in Production Example 1 except that 2-(7,12-di(3,5-di-tert-butylphenyl)benzo[k]fluoranthen-3-yl)-4,4,5,5-tetramethyl-1,3,2-dioxaborolane was used instead of 2-(7,12-diphenylbenzo[k]fluoranthen-3-yl)-4,4,5,5-tetramethyl-1,3,2-dioxaborolane.

MALDI-TOF MS was conducted to confirm 828.5, which was M⁺ of the compound. The structure of the compound was confirmed by ¹H-NMR spectroscopy.

¹H-NMR (CDCl₃, 400 MHz) δ (ppm): 8.06 (1H, bs), 7.99 (1H, d), 7.98 (1H, d), 7.94-7.84 (4H, m), 7.80-7.77 (2H, m), 7.67-7.60 (4H, m), 7.54 (1H, dd), 7.46-7.41 (7H, m), 7.30 (1H, t), 6.65 (1H, d), 6.59 (1H, d), 1.42 (36H, s)

The PL spectrum of the toluene solution containing Example Compound 2-5 was measured as in Production Example 1. A blue emission spectrum having the maximum intensity at 452 nm was detected.

### Production Examples 3 and 4

### [Method for Making Example Compounds 2-6 and 2-15]

Example Compounds described below were synthesized as in Production Example 1 except that 8-chlorofluoranthene was replaced by the following compounds.

### Production Example 3

### Example Compound 2-6: 2,5-di-tert-butyl-8-chlorofluoranthene

MALDI-TOF MS was conducted to confirm 716.3, which was M⁺ of the compound. The structure of the compound was confirmed by ¹H-NMR spectroscopy.

¹H-NMR (CDCl₃, 500 MHz) δ (ppm): 8.06 (2H, s), 8.01 (1H, d), 8.00 (1H, s), 7.95 (1H, d), 7.81 (2H, s), 7.71-7.59 (12H, m), 7.52 (1H, d), 7.46 (1H, d), 7.42-7.40 (2H, m), 7.35 (1H, t), 6.70 (1H, d), 6.67 (1H, d), 1.52 (9H, s), 1.47 (9H, s)

The PL spectrum of the toluene solution containing Example Compound 2-6 was measured as in Production Example 1. A blue emission spectrum having the maximum intensity at 449 nm was detected.

### Production Example 4

### Example Compound 2-15: 1-methyl-8-chlorofluoranthene

MALDI-TOF MS was conducted to confirm 618.2, which was M⁺ of the compound. The structure of the compound was confirmed by ¹H-NMR spectroscopy.

¹H-NMR (CDCl₃, 500 MHz) δ (ppm): 8.09 (1H, s), 8.07 (1H, d), 7.93 (2H, d), 7.83 (1H, d), 7.77 (1H, d), 7.72-7.54 (14H, m), 7.46 (2H, d), 7.42-7.40 (2H, m), 7.34 (1H, t), 6.71 (1H, d), 6.67 (1H, d), 2.94 (3H, s)

The PL spectrum of the toluene solution containing Example Compound 2-15 was measured as in Production Example 1. A blue emission spectrum having the maximum intensity at 450 nm was detected.

### Production Example 5

### [Method for Making Example Compound He-3]

Example Compound He-3, which is one example of the organic compound represented by general formula [3] contained in the organic compound layer of the organic light-emitting device, can be made by the following method, for example.

### (1) Synthesis of Intermediate 1

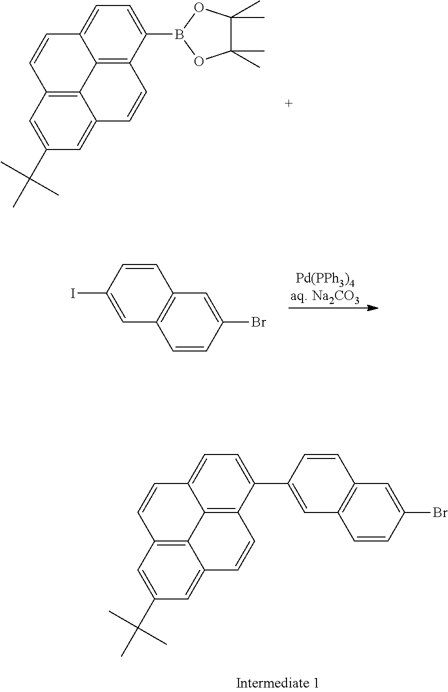

In a nitrogen atmosphere, compounds below were dissolved in a mixed solvent of toluene (70 ml) and ethanol (35 ml), an aqueous solution of 3.50 g (33.0 mmol) of sodium carbonate dissolved in 35 ml of distilled water was added to the resulting solution, and the resulting mixture was stirred for 3 hours in a silicone oil bath heated to 90° C. under heating: 4,4,5,5-tetramethyl-2-(7-tert-butylpyren-1-yl)-1,3,2-dioxaborolane 2.70 g (7.02 mmol); 2-bromo-6-iodonaphthalene 2.57 g (7.72 mmol); and tetrakis(triphenylphosphine)palladium 0.41 g (0.35 mmol).

The resulting mixture was cooled to room temperature and the organic layer was separated. The water layer was extracted (twice) with a mixed solvent of toluene and ethyl acetate and added to the solution of the organic layer separated initially. The organic layer was washed with saturated saline and dried with sodium sulfate. The solvent was distilled off and the residue was purified by silica gel column chromatography (toluene/heptane (1:30) mobile phase). The purified residue was vacuum dried at 80° C. As a result, 2.23 g (yield: 89%) of Intermediate 1 was obtained as a white solid.

MALDI-TOF MS was conducted to confirm 462.1, which was M⁺ of the compound. The structure of the compound was confirmed by ¹H-NMR spectroscopy.

¹H-NMR (CDCl₃, 500 MHz) δ (ppm): 8.25-8.20 (3H, m), 8.14-8.12 (2H, m), 8.09 (2H, m), 8.05 (1H, s), 8.02-8.00 (2H, m), 7.93 (1H, d), 7.82-7.79 (2H, m), 7.63 (1H, dd), 1.58 (9H, s)

### (2) Synthesis of Example Compound He-3

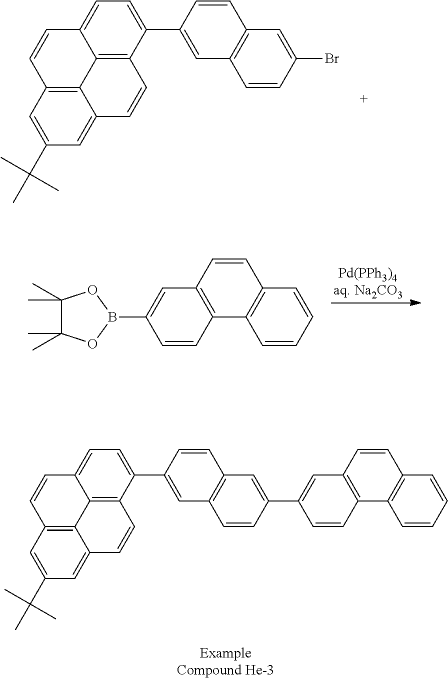

In a nitrogen atmosphere, compounds below were dissolved in a mixed solvent of toluene (16 ml) and ethanol (8 ml), an aqueous solution of 0.80 g (7.55 mmol) of sodium carbonate dissolved in 8 ml of distilled water was added to the resulting solution, and the resulting mixture was stirred for 3.5 hours in a silicone oil bath heated to 90° C. under heating: Intermediate 1 500 mg (1.08 mmol); 4,4,5,5-tetramethyl-2-phenanthren-2-yl-1,3,2-dioxaborolane 361 mg (1.19 mmol); and tetrakis(triphenylphosphine)palladium 62.3 mg (0.05 mmol).

The resulting mixture was cooled to room temperature and the organic layer was separated. The water layer was extracted (twice) with a mixed solvent of toluene and ethyl acetate and added to the solution of the organic layer separated initially. The organic layer was washed with saturated saline and dried with sodium sulfate. The solvent was distilled off and the residue was purified by silica gel column chromatography (toluene/heptane (1:10) mobile phase). The purified residue was further purified by recrystallization in a chloroform/ethanol (16:1) mixed solvent. A white solid obtained thereby was vacuum dried at 120° C. As a result, 500 mg (yield: 83%) of Example Compound He-3 was obtained.

MALDI-TOF MS was conducted to confirm 560.3, which was M⁺ of the compound. The structure of the compound was confirmed by ¹H-NMR spectroscopy.

¹H-NMR (CDCl₃, 500 Hz) δ (ppm): 8.84 (1H, d), 8.76 (1H, d), 8.36 (1H, s), 8.31 (1H, s), 8.26-8.22 (4H, m), 8.16-8.07 (7H, m), 8.05-8.02 (2H, m), 7.94 (1H, d), 7.90-7.82 (3H, m), 7.70 (1H, t), 7.64 (1H, t), 1.60 (9H, s)

Note that Example Compound He-3 can also be synthesized by a production scheme in which a 7-tert-butylpyrenyl group is introduced after introduction of a phenanthryl group into 2-bromo-6-iodonaphthalene.

Example Compound He-3 thus synthesized was used to prepare a 0.1 wt % chloroform solution. The solution was dropped onto a glass plate and spin-coating was conducted first for 10 seconds at 500 rpm and then 40 seconds at 1000 rpm to form a film on the glass plate.

The absorption spectrum of the organic film was measured with V-560 produced by JASCO Corporation to calculate the energy gap of Example Compound He-3. The value is shown in Table 1 below.

Note that the energy gap was calculated as the energy of the wavelength at the point of intersection between the wavelength axis and the tangent line drawn to the long wavelength-side absorption edge of the absorption spectrum.

### Production Examples 6 to 12

[Method for making Examples Compounds He-1, He-2, He-4, He-5, Hf-1, Hf-2, and Hf-3]

Examples Compounds below were synthesized as in Production Example 5 except that the following compounds were used instead of 4,4,5,5-tetramethyl-2-phenanthren-2-yl-1,3,2-dioxaborolane of (2) in Production Example 5:
Production Example 6 (Example Compound He-1): 4,4,5,5-tetramethyl-2-naphthalen-2-yl-1,3,2-dioxaborolane;
Production Example 7 (Example Compound He-2): 4,4,5,5-tetramethyl-2-(9,9-dimethylfluoren-2-yl)-1,3,2-dioxaborolane;
Production Example 8 (Example Compound He-4): 4,4,5,5-tetramethyl-2-phenanthren-9-yl-1,3,2-dioxaborolane; and
Production Example 9 (Example Compound He-5): 4,4,5,5-tetramethyl-2-(7-tert-butylpyren-1-yl)-1,3,2-dioxaborolane.

Example Compounds below were synthesized as in Production Example 5 except that 2-bromo-7-iodo-9,9-dimethylfluorene was used instead of 2-bromo-6-iodonaphthalene in (1) of Production Example 5 and the following compounds were used instead of 4,4,5,5-tetramethyl-2-phenanthren-2-yl-1,3,2-dioxaborolane of (2) in Production Example 5:
Production Example 10 (Example Compound Hf-1): 4,4,5,5-tetramethyl-2-naphthalen-2-yl-1,3,2-dioxaborolane;
Production Example 11 (Example Compound Hf-2): 4,4,5,5-tetramethyl-2-(9,9-dimethylfluoren-2-yl)-1,3,2-dioxaborolane; and
Production Example 12 (Example Compound Hf-3): 4,4,5,5-tetramethyl-2-(7-tert-butylpyren-1-yl)-1,3,2-dioxaborolane.

The energy gap of the resulting compounds were calculated as in Production Example 5. The values are shown in Table 1 below:

TABLE 1

| | Compound | Energy gap/eV |
|---|---|---|
| Production | Example Compound | 2.99 |
| Example 5 | He-3 | |
| Production | Example Compound | 2.99 |
| Example 6 | He-1 | |
| Production | Example Compound | 3.01 |
| Example 7 | He-2 | |
| Production | Example Compound | 2.98 |
| Example 8 | He-4 | |
| Production | Example Compound | 2.96 |
| Example 9 | He-5 | |
| Production | Example Compound | 3.01 |
| Example 10 | Hf-1 | |
| Production | Example Compound | 3.02 |
| Example 11 | Hf-2 | |
| Production | Example Compound | 2.95 |
| Example 12 | Hf-3 | |

### Example 1

An organic light-emitting device was prepared by the method described below.

A glass substrate with a thin film 120 nm in thickness formed by sputtering indium tin oxide (ITO) was used as a transparent conductive supporting substrate. The transparent conductive supporting substrate was ultrasonically washed with acetone and then isopropyl alcohol (IPA), washed with pure water, dried, and subjected to UV/ozone washing before use.

A 0.1 wt % chloroform solution was prepared by using Compound A as a hole transport material, Compound A being represented by the following structural formula:

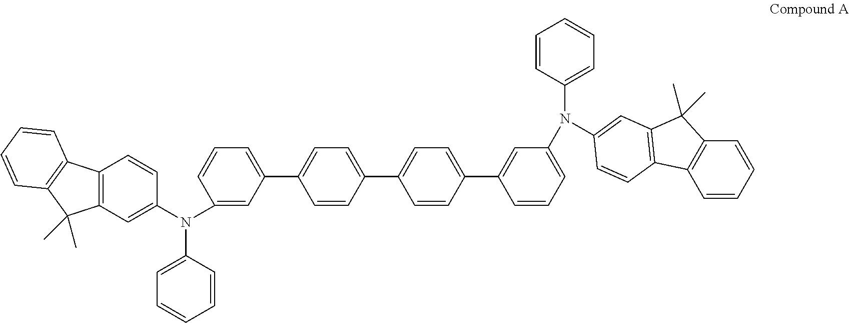

The solution was dropped onto the ITO electrode and spin-coating was conducted first for 10 seconds at 500 rpm and then 40 seconds at 1000 rpm to form a film. The film was dried in a vacuum oven at 80° C. for 10 minutes to completely remove the solvent in the thin film, thereby making a hole transport layer.

Next, Example Compound 1-1 and Example Compound He-1 (5:95 on a weight basis) were co-deposited on the hole transport layer to form an emission layer having a thickness of 30 nm. The degree of vacuum during deposition was 1.0×10⁻⁴ Pa and the deposition rate was 0.1 nm/sec to 0.2 nm/sec.

Then a film having a thickness of 30 nm was formed as an electron transport layer by vapor-depositing 2,9-bis[2-(9,9′-dimethylfluorenyl)]-1,10-phenanthroline. The degree of vacuum during deposition was 1.0×10⁻⁴ Pa and the deposition rate was 0.1 nm/sec to 0.2 nm/sec.

Next, lithium fluoride (LiF) was vacuum-deposited on the organic layer to form a film having a thickness of 0.5 nm, and an aluminum film having a thickness of 100 nm and serving as an electron injection electrode was formed thereon by vacuum deposition to obtain an organic light-emitting device. The degree of vacuum during deposition was 1.0×10⁻⁴ Pa. The deposition rate was 0.01 nm/sec for lithium fluoride and 0.5 nm/sec to 1.0 nm/sec for aluminum.

The resulting organic light-emitting device was covered with a protective glass plate in a dry air atmosphere and sealed with an acryl resin adhesive to prevent deterioration of the device by adsorption of moisture.

A voltage was applied to the device using the ITO electrode as the positive electrode and the Al electrode as the negative electrode. As a result, good blue emission having an emission efficiency of 3.9 cd/A at 1000 cd/m² was detected.

### Examples 2 to 12

Devices were prepared as in Example 1 except that Example Compounds shown in Table 2 were used instead of Example Compound 1-1 as the guest material and Example Compound He-1 as the host material.

When a voltage was applied to each of the devices of Examples, good blue emission was detected from all the devices. The emission efficiency at 1000 cd/m² is shown in Table 2 below.

TABLE 2

| | | | Emission |
|---|---|---|---|
| | Guest | | efficiency |
| | material | Host material | (cd/A) |
| Example 2 | Example | Example | 3.7 |
| | Compound 1-1 | Compound He-2 | |
| Example 3 | Example | Example | 4.1 |
| | Compound 1-1 | Compound He-3 | |
| Example 4 | Example | Example | 3.5 |
| | Compound 1-1 | Compound Hf-1 | |
| Example 5 | Example | Example | 3.7 |
| | Compound 1-1 | Compound Hf-2 | |
| Example 6 | Example | Example | 3.4 |
| | Compound 2-5 | Compound He-1 | |
| Example 7 | Example | Example | 3.8 |
| | Compound 2-6 | Compound He-3 | |
| Example 8 | Example | Example | 3.7 |
| | Compound 2-6 | Compound He-4 | |
| Example 9 | Example | Example | 4.4 |
| | Compound 2-6 | Compound He-5 | |
| Example 10 | Example | Example | 3.5 |
| | Compound 2-15 | Compound He-2 | |
| Example 11 | Example | Example | 3.2 |
| | Compound 2-15 | Compound He-3 | |
| Example 12 | Example | Example | 3.9 |
| | Compound 2-15 | Compound Hf-3 | |

### Example 13

An ITO film, a hole transport layer, and an emission layer were formed sequentially on a glass substrate as in Example 1.

Compound B was vacuum-deposited on the emission layer to form a film 10 nm in thickness serving as a hole blocking layer. The degree of vacuum during deposition was 1.0×10⁻⁴ Pa and the deposition rate was 0.1 nm/sec to 0.2 nm/sec:

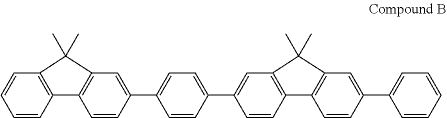

A film having a thickness of 20 nm was formed as an electron transport layer by vapor-depositing 2,9-bis[2-(9,9′-dimethylfluorenyl)]-1,10-phenanthroline. The degree of vacuum during deposition was 1.0×10⁻⁴ Pa and the deposition rate was 0.1 nm/sec to 0.2 nm/sec.

Next, lithium fluoride (LiF) was vacuum deposited on the organic layer to form a film having a thickness of 0.5 nm, and an aluminum film having a thickness of 100 nm and serving as an electron injection electrode was formed thereon by vacuum deposition to obtain an organic light-emitting device. The degree of vacuum during deposition was 1.0×10⁻⁴ Pa. The deposition rate was 0.01 nm/sec for lithium fluoride and 0.5 nm/sec to 1.0 nm/sec for aluminum.

The resulting organic light-emitting device was covered with a protective glass plate in a dry air atmosphere and sealed with an acryl resin adhesive to prevent deterioration of the device by adsorption of moisture.

A voltage was applied to the device using the ITO electrode as the positive electrode and the Al electrode as the negative electrode. As a result, good blue emission having an emission efficiency of 5.0 cd/A at 1000 cd/m² was detected.

### Examples 14 to 22

Devices were prepared as in Example 13 except that Example Compounds shown in Table 3 were used instead of Example Compound 1-1 as the guest material, Example Compound He-1 as the host material, and Compound B as the hole blocking material.

When a voltage was applied to each of the devices of Examples, good blue emission was detected from all the devices. The emission efficiency at 1000 cd/m² is shown in Table 3 below:

TABLE 3

| | | | Hole | Emission |
|---|---|---|---|---|
| | Guest | | blocking | efficiency |
| | material | Host material | material | (cd/A) |
| Example | Example | Example | Compound | 5.0 |
| 14 | Compound 2-5 | Compound Hf-2 | B | |
| Example | Example | Example | Compound | 5.3 |
| 15 | Compound 2-6 | Compound Hf-3 | B | |
| Example | Example | Example | Compound | 5.1 |
| 16 | Compound 2-15 | Compound He-1 | B | |
| Example | Example | Example | Compound | 5.0 |
| 17 | Compound 1-1 | Compound He-3 | C | |
| Example | Example | Example | Compound | 4.8 |
| 18 | Compound 2-5 | Compound He-4 | C | |
| Example | Example | Example | Compound | 4.9 |
| 19 | Compound 2-6 | Compound He-2 | D | |
| Example | Example | Example | Compound | 4.7 |
| 20 | Compound 2-15 | Compound He-5 | D | |
| Example | Example | Example | Compound | 5.1 |
| 21 | Compound 1-1 | Compound Hf-1 | E | |
| Example | Example | Example | Compound | 4.7 |
| 22 | Compound 2-5 | Compound He-1 | E | |

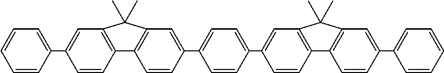

Compound C 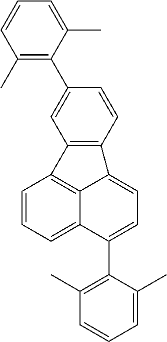
Compound D 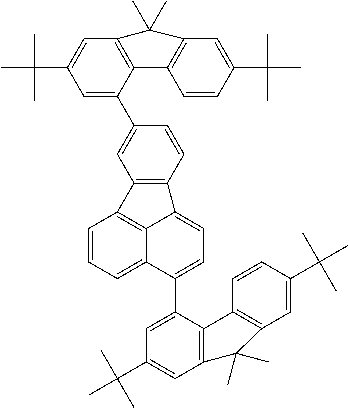
Compound E

### Comparative Example 1

Compound G1 was used as a comparative example to compare thermal stability:

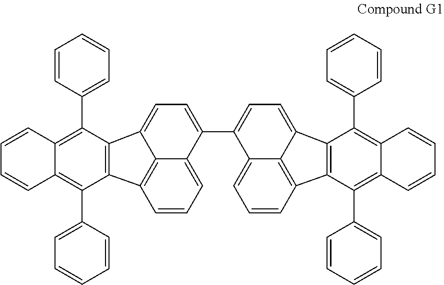

Example Compound 1-1 used in a light-emitting device of the present invention and Compound G1 serving as a comparative example were heated at a degree of vacuum of 2.0×10⁻¹ Pa up to 360° C. Compound G1 gradually turned red, and an emission peak derived from Compound G2 was detected. Example Compound 1-1 melted and turned yellow; however, no additional compound was confirmed by analysis after cooling.

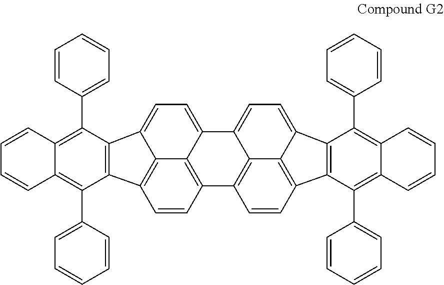

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2008-272133, filed Oct. 22, 2008, and Japanese Patent Laid-Open No. 2009-210707, filed Sep. 11, 2009, which are hereby incorporated by reference herein in their entirety.

1. An organic light-emitting device comprising:
an anode;
a cathode; and
an emission layer disposed between the anode and the cathode,
wherein the emission layer contains a compound represented by general formula [1] below and a compound represented by general formula [2] below: 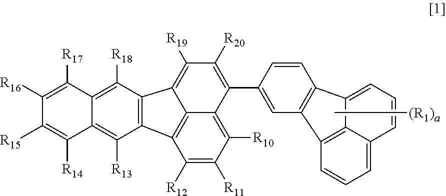
(in general formula [1], a represents an integer in the range of 0 to 9; R₁s each represent a group selected from a hydrogen atom, a halogen atom, a substituted or unsubstituted alkyl group, a substituted or unsubstituted aralkyl group, a substituted or unsubstituted aryl group, and a substituted or unsubstituted heterocyclic group and may be the same as or different from each other; and R₁₀ to R₂₀ each represent a group selected from a hydrogen atom, a halogen atom, a substituted or unsubstituted alkyl group, a substituted or unsubstituted aralkyl group, a substituted or unsubstituted aryl group, and a substituted or unsubstituted heterocyclic group and may be the same as or different from each other) 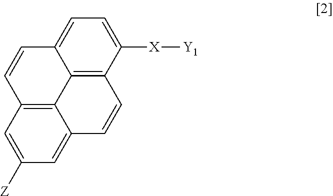
{in general formula [2], X represents a divalent substituted or unsubstituted fused ring aromatic group in which the number of fused rings is 2 or 3; Y₁ represents a group selected from a substituted or unsubstituted aryl group and a substituted or unsubstituted heterocyclic group; and Z represents a substituent represented by general formula [A] below: 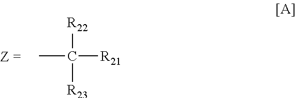
(in general formula [A], at least two of R₂₁ to R₂₃ represent substituted or unsubstituted alkyl groups and the remaining substituent is a hydrogen atom; and R₂₁ to R₂₃ may be the same as or different from each other.)}

2. The organic light-emitting device according to claim 1, wherein, in general formula [1], when a is 1 or more, one or more R₁s represent substituted or unsubstituted alkyl groups which may be the same as or different from each other, R₁₃ and R₁₈ among R₁₀ to R₂₀ each represent an unsubstituted phenyl group, and the compound represented by general formula [2] is a compound represented by general formula [3] below: 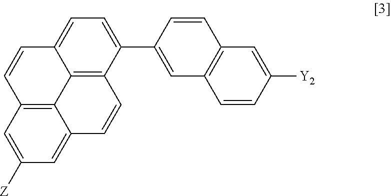
(in general formula [3], Y₂ represents a group selected from a 2-naphthyl group, a 2-(9,9-dimethyl)fluorenyl group, a 2-phenanthryl group, a 9-phenanthryl group, and a 1-(7-tert-butyl)pyrenyl group.)

3. The organic light-emitting device according to claim 1,
wherein, in general formula [1], when a is 1 or more, one or more R₁s represent substituted or unsubstituted alkyl groups which may be the same as or different from each other, R₁₃ and R₁₈ among R₁₀ to R₂₀ each represent an unsubstituted phenyl group, and the compound represented by general formula [2] is a compound represented by general formula [4] below: 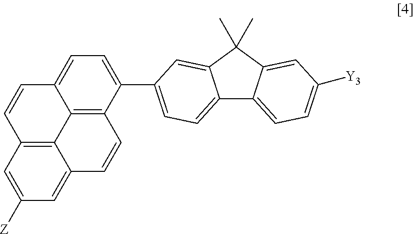
(in general formula [4], Y₃ represents a group selected from a 2-naphthyl group, a 2-(9,9-dimethyl)fluorenyl group, and a 1-(7-tert-butyl)pyrenyl group.)

4. A display apparatus comprising:
the organic light-emitting device according to claim 1; and
a switching device for controlling luminance of emission from the organic light-emitting device.
